SEQUENCE LISTING (15 sequences)

SEQ ID NO: 1 - protein, 9 aa, synthetic construct
  REGION 1..9: note = Synthetic: Biotinylated precision peptide
  MOD_RES 1: note = Biotin
CSPGAKVRC

SEQ ID NO: 2 - DNA, 27 bp, synthetic construct
  misc_feature 1..27: note = Synthetic: CD206 target
  source 1..27: mol_type = other DNA
tgctctccgggggcgaaagtcaggtgc

SEQ ID NO: 3 - DNA, 2022 bp, synthetic construct
  misc_feature 1..2022: note = Synthetic: Lamp2b signal peptide - linker - CD206 target- linker- mIgG2b -linker- Lamp2b c-terminus sequence - 6His-tag - stop codon
  source 1..2022: mol_type = other DNA
atgtgcctctctccggttaaaggcgcaaagctcatcctgatctttctgttcctaggagccgttcagtccaatgcagcgcgatgctctccgggggcgaaagtcaggtgcgctcgtgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtagcggaggcttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtccaccaccaccaccaccactaa

SEQ ID NO: 4 - DNA, 75 bp, synthetic construct
  misc_feature 1..75: note = Synthetic: Lamp2b signal peptide
  source 1..75: mol_type = other DNA
atgtgcctctctccggttaaaggcgcaaagctcatcctgatctttctgttcctaggagccgttcagtccaatgca

SEQ ID NO: 5 - DNA, 1173 bp, synthetic construct
  misc_feature 1..1173: note = Synthetic: Lamp2b c-terminus sequence
  source 1..1173: mol_type = other DNA
ttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtc

SEQ ID NO: 6 - DNA, 705 bp, synthetic construct
  misc_feature 1..705: note = Synthetic: mIgG2b
  source 1..705: mol_type = other DNA
gggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggt

SEQ ID NO: 7 - protein, 6 aa, synthetic construct
  REGION 1..6: note = Synthetic: 6xHis-tag
HHHHHH

SEQ ID NO: 8 - DNA, 1845 bp, synthetic construct
  misc_feature 1..1845: note = Synthetic: Lamp2b signaling-linker-RGV-linker-NGB-linker-Myc-linker-Lamp2b c-terminus-Stop codon
  source 1..1845: mol_type = other DNA
atgtgcctctctccggttaaaggcgcaaagctcatcctgatctttctgttcctaggagccgttcagtccaatgcaagcgcttacaccatctggatgcccgagaaccccaggcccggcaccccctgcgacatcttcaccaacagcaggggcaagagggccagcaacggcagcgctatggagcgcccggagtcagagctgatccggcagagctggcgggtagtgagccgcagccctctggaacatggcactgtcctgttcgccaggctcttcgccctggaacccagcctgctgcctctcttccagtacaatggccgccagttctccagccctgaggactgtctctcctctccagaattcctggaccacattaggaaggtgatgctagtgattgatgctgcagtgaccaacgtggaggacctgtcttcattggaggagtacctgaccagcttgggcaggaagcatcgggcagtgggagtgaggctcagctccttctcgacagtaggcgagtccctgctctacatgctggagaagtgcctgggtcccgactttacaccagctacaaggaccgcctggagccgactctacggagctgtggtgcaagccatgagccgaggctgggatggggagggatccgagcagaaactcatctctgaagaggatctgcccgggttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtctaa

SEQ ID NO: 9 - DNA, 2925 bp, synthetic construct
  misc_feature 1..2925: note = Synthetic: Lamp2b signaling-linker-RGV-linker-Myc-linker-Lamp2b c-terminus-EcoRV-T2A-WW tag-NGB-P2A-Ndfib1-EcoRV-Stop codon
  source 1..2925: mol_type = other DNA
atgtgcctctctccggttaaaggcgcaaagctcatcctgatctttctgttcctaggagccgttcagtccaatgcaagcgcttacaccatctggatgcccgagaaccccaggcccggcaccccctgcgacatcttcaccaacagcaggggcaagagggccagcaacggcggatccgagcagaaactcatctctgaagaggatctgcccgggttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtcgatatcggaagcggagagggcaggggaagtcttctaacatgcggggacgtggaggaaaatcccggccccatgagcggcctgccccccggctgggaggagaagcaggacgacaggggcaggagctactacgtggaccacaacagcaagaccaccacctggagcaagcccaccatgcaggacgaccccaggagcaagatccccgcccacctgaggggcaagaccgacagcaacgacctgggccccctgccccccggctgggaggagaggacccacaccgacggcagggtgttcttcatcaaccacaacatcaagaagacccagtgggaggaccccaggctgatggagcgcccggagtcagagctgatccggcagagctggcgggtagtgagccgcagccctctggaacatggcactgtcctgttcgccaggctcttcgccctggaacccagcctgctgcctctcttccagtacaatggccgccagttctccagccctgaggactgtctctcctctccagaattcctggaccacattaggaaggtgatgctagtgattgatgctgcagtgaccaacgtggaggacctgtcttcattggaggagtacctgaccagcttgggcaggaagcatcgggcagtgggagtgaggctcagctccttctcggtgggtagtgggacagtaggcgagtccctgctctacatgctggagaagtgcctgggtcccgactttacaccagctacaaggaccgcctggagccgactctacggagctgtggtgcaagccatgagccgaggctgggatggggagggaagcggagccacgaacttctctctgttaaagcaagcaggagatgttgaagaaaaccccgggcctatggccttggcgttggcggcgctggcggccgtggagccggcctgcggcagcgggtaccagcagttgcagaatgaagaggagcctggggaacctgagcagactgcaggtgatgctcctccaccatacagcagcatcacggcagagagtgcagcatattttgactacaaagatgaatctggatttccaaagcccccatcgtataatgtggctacaacactgcccagttatgacgaggctgagagaaccaagactgaagctacgatccctttggttcctggaagagatgaagattttgtgggccgggatgattttgatgatactgaccagctgaggataggaaacgatgggatttttatgttaacttttttcatggcattcctcttcaactggattgggtttttcttgtctttttgcctgaccacctcagctgcgggaaggtatggggccatctcaggatttggtctttctctaattaagtggatccttattgtcaggttttccacctatttccctggatactttgatggccagtactggctctggtgggtgttcttggttttaggctttctcctgtttctcagaggatttatcaattacgcaaaagttcggaagatgccagaaactttctcaaatctccccaggaccagagttctctttatttatgatatctaa

SEQ ID NO: 10 - DNA, 2451 bp, synthetic construct
  misc_feature 1..2451: note = Synthetic: Lamp2b signaling peptide-linker-mouse CSF1R targeting peptides-linker-mouse IgG2b- linker-Myc-Lamp2b C-terminus-Stop codon
  source 1..2451: mol_type = other DNA
atgtgcctctctccggttaaaggcgcaaagctcatcctgatctttctgttcctaggagccgttcagtccaatgcaagcgctggtaactcgactatgggcagtggatcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaggcagtggatctggtggctcgagtggatccgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtgatatcggagagcagaaactcatctctgaagaggatctgttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtccatcaccatcaccatcactaa

SEQ ID NO: 11 - DNA, 36 bp, synthetic construct
  misc_feature 1..36: note = Synthetic: EPHA2 targeting peptide
  source 1..36: mol_type = other DNA
tacagcgcctaccccgacagcgtgcccatgatgagc

SEQ ID NO: 12 - protein, 6 aa, synthetic construct
  REGION 1..6: note = Synthetic: antagonistic peptide
FTFEPF

SEQ ID NO: 13 - DNA, 18 bp, synthetic construct
  misc_feature 1..18: note = Synthetic: nucleotide encoding antagonistic peptide
  source 1..18: mol_type = other DNA
ttcaccttcgagcccttc

SEQ ID NO: 14 - protein, 11 aa, synthetic construct
  REGION 1..11: note = Synthetic: antagonistic peptide
ETYIKPWWVWL

SEQ ID NO: 15 - DNA, 33 bp, synthetic construct
  misc_feature 1..33: note = Synthetic: nucleotide encoding antagonistic peptide
  source 1..33: mol_type = other DNA
gagacctacatcaagccctggtgggtgtggctg

(19) United States
(12) Patent Application Publication
(10) Pub. No.: US 2024/0075161 A1
(43) Pub. Date: Mar. 7, 2024

(54) ENGINEERED EXOSOMES TO DETECT AND DEPLETE PRO-TUMORIGENIC MACROPHAGES

(71) Applicant: AUGUSTA UNIVERSITY RESEARCH INSTITUTE, INC., Aigusta, GA (US)

(72) Inventors: Ali ARBAB, Augusta, GA (US); Mohammad Harun RASHID, Augusta, GA (US); Thaiz BORIN, Augusta, GA (US); Roxan ARA, Augusta, GA (US)

(73) Assignee: AUGUSTA UNIVERSITY RESEARCH INSTITUTE, INC., Augusta, GA (US)

(21) Appl. No.: 18/500,774

(22) Filed: Nov. 2, 2023

Related U.S. Application Data
(63) Continuation-in-part of application No. 17/083,124, filed on Oct. 28, 2020, now abandoned.
(60) Provisional application No. 62/926,775, filed on Oct. 28, 2019.

(51) Int. Cl. A61K 47/69; A61K 35/22; A61K 47/62; C07K 14/47; G01N 33/58

(52) U.S. Cl. CPC A61K 47/6901; A61K 35/22; A61K 47/62; C07K 14/47; G01N 33/58; C07K 2319/30

(57) ABSTRACT

An engineered targeting exosome comprising; a modified Lamp2b peptide, a targeting peptide or antibody; and a Fc portion of IgG2b. The targeting peptide or antibody detects a target protein within a cell and precisely delivers the exosome to cells expressing the target protein and the Fc portion of IgGb2 induces antibody-dependent cell-mediated cytotoxicity (ADCC) events in cells expressing the target protein. Also disclosed are methods of using the engineered targeting exosome to detect and/or deplete cells expressing the target protein.

### REFERENCE TO RELATED APPLICATIONS

This application is a continuation-in-part of U.S. patent application Ser. No. 17/083,124, filed on Oct. 28, 2020, which claims benefit of and priority to U.S. Provisional Application No. 62/926,775 filed on Oct. 28, 2019, each of which are incorporated by reference in their entirety.

### ACKNOWLEDGMENT OF GOVERNMENT SUPPORT

This invention was made with government support under RO1 CA160216 awarded by the National Institutes of Health. The government has certain rights in the invention.

### SEQUENCE LISTING

The application contains a Sequence Listing which has been submitted electronically in .XML format and is hereby incorporated by reference in its entirety. Said .XML copy, created on Oct. 31, 2023, is named “064466.119CIP.xml” and is 31,836 bytes in size. The sequence listing contained in this .XML file is part of the specification and is hereby incorporated by reference herein in its entirety.

### FIELD

Aspects of the invention are generally directed to compositions and methods of engineered exosomes.

### BACKGROUND

Exosomes have emerged as potential tools for a drug delivery system that can target specific tissues or cells. Recently, the therapeutic application of exosomes has shown promising results as novel therapeutic vehicles in cancer immunotherapy and suicide therapy, as well as delivery of RNA-interference and drugs (Yu et al., J. Immunol. 2007, 178, 6867-6875; El Andaloussi et al., Adv. Drug Deliv. Rev. 2013, 65, 391-397; El Andaloussi et al., Nat. Protocols 2012, 7, 2112-2126; Chaput et al., Cancer Immunol., Immunother.: CII 2004, 53, 234-239; Kurywchak et al., Genome Med. 2018, 10, 23 (2018)). Exosomes have clear advantages over synthetic nanoparticles like liposomes as a vehicle because of their improved biocompatibility, low toxicity and immunogenicity, permeability, stability in biological fluids, and ability to accumulate in the tumor with higher specificity (Mager et al, Nat. Rev. Drug Discov. 2013, 12, 347-357; Lener et al., J. Extracell. Vesicles 2015, 4, 30087; Jiang et al., Int. J. Pharm. 2017, 521, 167-175; Alvarez-Erviti et al., Nat. Biotechnol. 2011, 29, 341-345). Exosomes can be engineered to express targeting peptides or antibodies on their surface for precise targeted therapeutics delivery (Morishita et al., Biomaterials 2016, 111, 55-65; Stickney et al., Biochem. and Biophys. Res. Commun. 2016, 472, 53-59; Yim et al.; Nat Commun. 2016, 7, 12277).

Antibody-dependent cell-mediated cytotoxicity (ADCC) is a non-phagocytic mechanism by which most leucocytes (effector cells) can kill antibody-coated target cells in the absence of complement and without major histocompatibility complex (MHC) (van Dommelen et al., J. Controlled Release 2012, 161, 635-644). Targeted therapy utilizing monoclonal antibodies (mAbs) has instituted immunotherapy as a robust new tool to fight against cancer. As mAb therapy has revolutionized treatment of several diseases, ADCC has become more applicable in a clinical context. Clinical trials have demonstrated that many mAbs perform somewhat by eliciting ADCC (van der Meel et al., J. Controlled Release 2014, 195, 72-85). Antibodies serve as a bridge between Fc receptors (FcR) on the effector cell and the target antigen on the cell that is to be killed. There has not been any report of engineered targeted/therapeutic exosomes inducing ADCC. In the proposed model of engineered exosomes along with CD206 binding peptide, we conjugated Fc portion of the mouse IgG2b that could potentially be recognized by FcR on the effector cells and stimulate the ADCC events.

Therefore, it is an object of the invention to provide compositions of engineered exosomes for detecting and depleting targeted cells.

There has not been any report showing exosomes can initiate ADCC to kill targeted cells. Recent investigations pointed out that tumor-derived exosomes cause immunosuppression, inhibit the binding of tumor-reactive antibodies to tumor cells, and reduce ADCC (Steinbichler T B, Dudás J, Skvortsov S, Ganswindt U, Riechelmann H, Skvortsova I-I. Therapy resistance mediated by exosomes. Molecular Cancer. 2019; 18:58. doi: 10.1186/s12943-019-0970-x, Battke C, Ruiss R, Welsch U, Wimberger P, Lang S, Jochum S, Zeidler R. Tumour exosomes inhibit binding of tumour-reactive antibodies to tumour cells and reduce ADCC. Cancer Immunology, Immunotherapy. 2011; 60:639-648. doi: 10.1007/s00262-011-0979-5). Provided herein are exosomes designed using DNA technology to express a protein of interest either on the surface or inside the lumen of the exosomes. The provided exosome engineering technologies are used as a platform to insert or express any protein or peptide to target specific cells in the body or in the tumor and if needed, to deplete the cells by inserting depleting protein, such as IgG2b, used herein to deplete ADCC.

### SUMMARY

In accordance with the purpose(s) of this invention, as embodied and broadly described herein, this invention, in one aspect, relates to an engineered targeting exosome comprising; a Lamp2b signal peptide; a targeting peptide or antibody; a Fc portion of IgG2b; a c-terminus portion of Lamp2b protein; a tag protein at the C-terminus; and a reporter gene; wherein, the targeting peptide or antibody detects target protein within cells expressing the target protein and precisely delivers the exosome to the cells expressing the target protein; and wherein the Fc portion of IgGb2 induces antibody-dependent cell-mediated cytotoxicity (ADCC) events in cells expressing the target protein.

In one embodiment, the targeting peptide or antibody peptide or antibody is attached to the lumen or the surface of an exosome. In another embodiment, the targeting peptide or antibody is a fusion peptide or antibody comprises a flag tag, a therapeutic peptide, a targeting moiety, or other peptide or antibody attached to the peptide or antibody, or a modification or a fragment of the peptide or antibody. In yet another embodiment, the targeting peptide or antibody targets proteins involved in tumorigenesis and neurological disorders.

In another embodiment, the engineered targeting exosome penetrates through the blood brain barrier to target cells wherein the target protein is expressed. Where the targeting peptide or antibody targets proteins selected from a group consisting of CD206+ M2-macrophage, rabies virus glycoprotein (RVG), EPHA2, CSF-1R, and neutrophils (FPRs).

In one embodiment, the targeting peptide or antibody targets protein is encoded by nucleic acid sequences with 95%, 99% or more sequence identity to SEQ ID NO:2, 8, 9, 10, 11, 12 or 13. In another embodiment, the Lamp2b signal peptide is encoded by a nucleic acid sequence with 95%, 99% or more sequence identity to SEQ ID NO:4 and the Lamp2b c-terminus protein is encoded by a nucleic acid sequence with 95%, 99% or more sequence identity to SEQ ID NO:5. In other embodiment, the Fc portion of IgG2b is encoded by a nucleic acid sequence with 95%, 99% or more sequence identity to SEQ ID NO:6.

In some embodiments, the targeting exosome is loaded with cargo, wherein the cargo is selected from the group consisting of a detectable label, a chemotherapeutic agent, and a cytotoxic agent.

Other embodiments relate to pharmaceutical composition comprising the engineered targeting exosome of and a pharmaceutically acceptable excipient.

In another aspect, the invention relates to a method of depleting cells expressing a targeted protein in a subject in need thereof comprising administering to the subject an effective amount of a composition comprising an exosome engineered to express a targeting peptide or antibody and an Fc portion of IgGb2, wherein the targeting peptide or antibody targets cells expressing the target protein; and wherein the Fc portion of IgGb2 induces antibody-dependent cell-mediated cytotoxicity (ADCC) in the cells expressing the targeted protein.

In one embodiment, the subject is a mammal, wherein the subject has cancer, a neurological disorder, or another disease or disorder to be targeted.

In some embodiments, the exosomes are loaded with cargo, wherein the cargo is selected from the group consisting of a detectable label, a chemotherapeutic agent, and a cytotoxic agent.

In yet another aspect, the invention relates to a method for detecting targeted protein in cells expressing the targeted protein comprising: contacting a biological sample with a composition comprising an exosome engineered to express a targeting peptide or antibody, wherein the exosome is loaded with a detectable label; and detecting the detectable label, wherein the detection of the label indicates the presence of the of the targeted protein in the sample; wherein the exosome comprises a Lamp2b signal peptide; a targeting peptide or antibody; a c-terminus portion of Lamp2b protein; a tag protein at the C-terminus; and a reporter gene.

In one embodiment, the cells expressing the targeted protein are cancer cells, brain cells, bone marrow derived cells (such as macrophages and neutrophils) or cells of other diseases to be detected. In some embodiments, the exosome penetrates through the blood brain barrier to treat neurological disorders where the targeted protein is expressed.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIGS. 1A-1I represent generation of engineered exosomes expressing CD206-positive M2 macrophage-specific peptide along with Lamp2b.

FIG. 1A exhibits immunofluorescence staining of tumor, spleen and lungs sections from 4T1 tumor-bearing mice showing co-localization of Rhodamine red-labeled targeting peptide (injected i.v.) and FITC labeled CD206-positive M2-macrophages. Nuclei were visualized by DAPI staining (blue).

FIG. 1B is a schematic representation of the modified Lamp2b protein containing CD206 positive M2 macrophage-targeting peptide sequence following signal peptide, and a 6×HIS tag (SEQ ID NO:7) at the C terminus. Luciferase was used as a reporter gene.

FIG. 1C is a schematic diagram showing generation of CD206+ M2-macrophage targeting engineered exosomes for diagnostic and therapeutic purpose.

FIG. 1D represents in vitro study showing luciferase activity of transfected HEK293 cells.

FIG. 1E is agarose gel electrophoresis showing confirmation of targeting peptide sequence insert in transfected HEK293 cells.

FIG. 1F is a Western blot image showing anti-His tag antibody positivity in engineered exosomal protein content. Figure discloses SEQ ID NO:7.

FIG. 1G and FIG. 1H showing size distribution by nanoparticle tracking assay (NTA) of the HEK293 exosomes and engineered exosomes, respectively. Quantitative data are expressed in mean±SEM.

FIG. 1I illustrates a transmission electron microscopy image for engineered exosomes, (Scale bar depicts 200 nm) showing characteristic round morphology and size without any deformity.

FIGS. 2A-D represent targeting efficiency and specificity of CD206-positive M2 macrophage-specific exosomes.

FIG. 2A exhibits immunofluorescence staining showing targeting potential of DiI-labeled (red) engineered exosomes. RAW264.7 mouse macrophages were differentiated to CD206-positive (FITC) cells by treating with interleukin-4 and interleukin-13. Nuclei were visualized by DAPI staining (blue).

FIG. 2B exhibits immunofluorescence staining of mouse embryonic fibroblasts (MEFs) and RAW264.7 cells treated with or without anti-CD206 peptide, co-cultured with DiI-labeled (red) engineered exosomes. MEFs were negative for CD206 (FITC) staining and did not take up the exosomes. Engineered exosomes bound to the CD206+RAW264.7 cells that was prevented by anti-CD206 peptide treatment.

FIG. 2C exhibits immunofluorescence staining of tumor, spleen and lungs sections from 4T1 tumor-bearing mice showing co-localization of rhodamine red-labeled targeting exosomes (injected i.v.) and FITC labeled CD206-positive M2-macrophages. Nuclei were visualized by DAPI staining (blue).

FIG. 2D exhibits stitched images for extended view of splenic section showing engineered exosomes were not taken up by T-lymphocytes and B-lymphocytes in splenic white pulp (white arrows).

FIGS. 3A-G represent detection and quantification of biodistribution of 111In-oxine-labeled exosomes targeting CD206-positive M2 macrophages.

FIG. 3A shows a major proportion of the free 111In-oxine measured in the bottom to the top half of the thin layer paper chromatography (TLPC) paper, confirming the efficacy of the eluent.

FIG. 3B shows binding of 111In-oxine to engineered exosomes was validated as shown by a lower percentage of 111In-oxine (free, dissociated) measured in the top of the paper, compared to the amount remaining in the bottom, which represented the 111In-oxine-labeled exosomes.

FIG. 3C shows serum stability of 111In-oxine bound engineered exosomes was higher compared with the small amount of free 111In-oxine disengaged from the bound exosomes.

FIG. 3D illustrates in vivo SPECT/CT images (coronal view) after 3 hrs of intravenous injection showed significant accumulation of M2-targeting exo in tumor, lung, spleen, lymph node and bones. 111In-oxine-labeled non-targeting exosomes (HEK293 exo) and CD206-positive M2-macrophage targeting exosomes (M2-targeting exo) were injected into the 4T1 tumor-bearing mice. One group was treated with Clophosome® to deplete macrophages. Yellow and green arrows denote lymph node and bone metastasis, respectively.

FIG. 3E illustrates 3D surface images showing M2-targeting exo are profoundly distributed in both lung and tumor area compared to the group injected with HEK293 exo and pre-treated with Clophosome®. Yellow arrow indicates the tumor center.

FIG. 3F shows quantification of in vivo radioactivity in lungs, spleen and tumor.

FIG. 3G shows ex vivo radioactivity quantification in lungs, spleen and tumor. Quantitative data are expressed in mean±SEM. *P<0.05, **P<0.01, ***P<0.001, ****P<0.0001. n=3.

FIGS. 4A-H represent generation of CD206-positive M2 macrophage-targeting therapeutic exosomes to induce antibody-dependent cell-mediated cytotoxicity.

FIG. 4A illustrates schematic diagram showing the proposed mechanism of engineered exosome-based antibody-dependent cellular cytotoxicity.

FIG. 4B illustrates schematic representation of the plasmid construct containing modified Lamp2b protein with CD206-targeting sequence conjugated with Fc segment of mouse IgG2b. Figure discloses SEQ ID NO:7.

FIG. 4C demonstrates confirmation of luciferase activity by transfected HEK293 cells.

FIG. 4D shows flow cytometry analysis for validating the expression of Fc segment of mouse IgG2b on the surface of engineered exosomes. Three different engineered exosome samples were used for the flow cytometry.

FIG. 4E shows concentration and size distribution of the engineered therapeutic exosomes by nanoparticle tracking assay (NTA).

FIG. 4F shows mean diameter of engineered exosomes was significantly larger than non-engineered exosomes

FIG. 4G illustrates transmission electron microscopy image for engineered therapeutic exosomes, (Scale bar depicts 100 nm) showing distinctive round morphology and size without any distortion.

FIG. 4H shows flow-cytometry analysis of exosomal markers CD9 and CD63 for the engineered therapeutic exosomes. Three different engineered exosome samples were used for the flow cytometry.

FIGS. 5A-F represent therapeutic efficiency and specificity of engineered therapeutic exosomes in depleting M2-macrophages both in vitro and in vivo.

FIG. 5A illustrates CFSE-labeled (green) RAW264.7 mouse macrophages were co-cultured with non-therapeutic CD206-positive cell-targeting exosomes (LAMP-206 exo) or CD206-positive cell-targeting therapeutic exosomes (LAMP-206-IgG2b exo), and without treatment (control) for 48 hours in presence of splenic immune cells from normal mice. Fluorescence microscopic images showed decrease in cell number and increased floating dead cells in LAMP-206-IgG2b exo group compared to other groups.

FIG. 5B shows measured fluorescence intensity of the above-mentioned conditions showed significant decrease in LAMP-206-IgG2b exo group compared to other groups.

FIG. 5C and FIG. 5D exhibit normal Balb/c mice were treated with one, two or three doses of engineered therapeutic exosomes expressing Fc portion of mouse IgG2b. Flow-cytometry analysis of splenic cells showing dose-dependent decline of F4/80 and CD206-positive M2-macrophage population.

FIG. 5E and FIG. 5F illustrate flow-cytometry analysis of splenic cells showing no significant change in both CD4 and CD8-positive T-cell population after treating the mice with different doses of therapeutic exosomes. Quantitative data are expressed in mean±SEM. *P<0.05, **P<0.01, ***P<0.001, ****P<0.0001. n=5.

FIGS. 6A-E represents treatment of 4T1 tumor-bearing animals with therapeutic engineered exosomes prevent tumor growth and metastasis, and improve survival by depleting M2-macrophages.

FIG. 6A and FIG. 6B illustrate reconstructed and co-registered in vivo SPECT/CT images (coronal view) and quantification of subcutaneous syngeneic tumor-bearing animals (on the flank) injected with the 99mTc-labeled precision peptide after three hours. Group treated with therapeutic exosomes showed lesser level of radioactivity in tumor (yellow arrow) and spleen compared to untreated control group. Quantitative data are expressed in mean±SEM, *P<0.05. n=3.

FIG. 6C displays optical images of 4T1 tumor-bearing animals treated with engineered therapeutic exosomes (lower panel) or without treatment (control), showing decreased tumor growth in treated animals compared to control group. Metastatic foci in control group was detected (yellow arrows) as early as fourth week, whereas no metastasis was detected in treated animals after 6 weeks.

FIG. 6D illustrates quantification of optical density of the tumor area also showed decreased tumor growth in treated group compared to control group. Quantitative data are expressed in mean±SEM. n=3.

FIG. 6E shows Kaplan-Meier plot showing prolonged survival of the mice treated with therapeutic engineered exosomes.

FIG. 7 is a schematic of a representative plasmid used to produce CD206-positive M2 macrophage-targeting exosomes.

FIG. 8 shows the expression of GAPDH-normalized mRNA of Ngb in stroke and normal tissues.

FIG. 9 is a schematic of the development of neuron targeting engineered exosomes to deliver Ngb.

FIGS. 10A-10G show results from the delivery of engineered exosomes to stroke area using focused ultrasound. Exosomes were IV administered 24hrs after stroke soon after focal ultrasound and brain collected after 3 hrs.

FIGS. 11A-11I shows results from specifically targeting of neuron in the stroke area. Exosomes were IV administered 24hrs after stroke soon after focal ultrasound and brain collected after 3 hrs.

FIG. 12 is a schematic of the development of the MDSC-targeting engineered exosomes to target and kill MDSC by ADCC.

FIG. 13A is a schematic of the development of the CSF1R targeting Peptide (CSF1) engineered exosomes to target and kill myeloid cells by ADCC. FIGS. 13B-13F are images showing the uptake of engineered exosomes by CSF1R+ cells using anti-CSF1R antibody. FIGS. 13G-13H are fluorescent intensity images of CSF1R+ cells engineered exosomes with (FIG. 13G) and without (FIG. 13H) NK cells (splenocytes). Note the diminution of fluorescent activity due to the death of CSF1R+ cells. FIGS. 13I-13J are images of orthotopic AT3 breast cancer bearing animals treated with control HEK exosomes (FIG. 13I) or with CSF1R+ cell targeting engineered exosomes (FIG. 13J).

FIGS. 14A-14B are schematics showing the construct of plasmid (FIG. 14A) and mechanisms of targeting GMB by ADCC (FIG. 14B).

FIG. 15 is a bioluminescent image showing stable transfection of neutrophil targeting peptide exosome into HEK293 cells.

FIG. 16 is a graph showing the characteristics of neutrophil targeting peptides exosomes.

FIGS. 17A-17F are images showing in vitro depletion of GBM cells by targeting therapeutic EphA2 exosomes.

FIGS. 18A-18B show the effect of engineered exosomes depleting EpHA2 positive GBM cells.

FIGS. 19A-19B schematic showing the development of the neutrophil binding peptide exosome plasmid (FIG. 19A) and the mechanism of targeting neutrophils by ADCC (FIG. 19B).

FIG. 20 is a bioluminescent image showing the stable transfection of peptides targeting neutrophils in HEK293 cells.

FIGS. 21A-21C are graphs showing the characteristics of the FTFEPF antagonistic peptide (FIG. 21A), ETYIK (ETYIKPWWVWL) (SEQ ID NO: 14) antagonistic peptide (FIG. 21B) and FTFEPF+ETYIK antagonistic peptides (FIG. 21C).

FIG. 22A-22B are bar graphs showing results of in vitro studies showing depletion of activated neutrophils by therapeutic exosomes without activation (FIG. 22A) and with activation by PMA (FIG. 22B).

FIGS. 23A-23G show the in vivo effects of therapeutic exosomes in a stroke model.

FIGS. 24A-24F are images showing in vivo delivery of therapeutic exosomes to the site of stroke through BBB targeting granulocytes.

FIGS. 25A-25H are images of brains showing the effect of engineered exosomes depleting activated neutrophils on stroke volume (FIGS. 24A-25H and FIG. 25J) and BBB leakage (FIG. 25I).

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

As used herein, the term “pharmaceutical composition” means a mixture comprising a pharmaceutically acceptable active ingredient, in combination with suitable pharmaceutically acceptable excipients.

As used herein, the term “pharmaceutical formulation” means a composition in which different chemical substances, including the active drug, are combined to produce a final medicinal product. Examples of formulation include enteral formulations (tablets, capsules), parenteral formulations (liquids, lyophilized powders), or topical formulations (cutaneous, inhalable).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable vehicle” refers to a diluent, adjuvant, excipient or carrier with which a compound of the invention is administered.

The term “Subject” includes mammals such as humans The terms “human”, “patient” and “subject” are used interchangeably herein.

“Effective amount” means the amount of a compound of the invention that, when administered to a subject for treating a disease, is sufficient to effect such treatment for the disease. The ‘effective amount’ can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment’” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed to be at risk for a disorder including those described herein or that would benefit from the delivery of a composition including those described herein.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “extracellular vesicle” or “EV” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and can comprise various macromolecular payload either within the internal space, displayed on the external surface of the extracellular vesicle, and/or spanning the membrane. Said payload can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “exosome” refers to a cell-derived small (between 20-300 nm in diameter, more preferably 30-200 nm in diameter) vesicle comprising a membrane that encloses an internal space, and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof.

As used herein, the term “target protein” or “target peptide” refers to a protein or peptide that can be targeted to the lumen or the surface of an exosome. The target protein or peptide can be a non-mutant protein that is naturally targeted to an exosome lumen or surface, or a fragment or a modification of the non-mutant protein. The target protein can be a fusion protein containing a flag tag, a therapeutic peptide, a targeting moiety, or other peptide attached to the non-mutant protein or a modification or a fragment of the non-mutant protein. The target protein can comprise a modification such as myristoylation, prenylation, or palmitoylation, or comprise a soluble protein attached to the internal leaflet of the membrane by a linker.

As used herein, the term “cargo” refers to any agent which can be loaded into an exosome or engineered exosome. The cargo is selected from the group consisting of a detectable label, a chemotherapeutic agent, and a cytotoxic agent.

As used herein, the term “cargo protein” or cargo peptide” refers to any protein or peptide, or fragment or modification thereof, which can be loaded into an exosome or engineered exosome. Cargo proteins or peptide may include therapeutic peptides or proteins that act on a target (e.g. a target cell) that is contacted with the exosome. Cargo proteins may be a fusion protein comprising a targeting protein or peptide or fragment or modification thereof, as described above, such that the cargo fusion protein can be targeted to an exosome lumen.

### II. Targeting Exosome Compositions

### A. Fc Gamma-Receptor (FcR) Based Targeting/Therapeutic Exosome Platform

In recent years, several pioneers have explored the possibility of using exosomes as drug delivery vehicles. Owing to their defined size and natural function, exosomes appear ideal candidates for theranostic nanomedicine application. When compared to the administration of free drugs or therapeutics, exosomes have certain advantages such as improved stability, solubility, and in vivo pharmacokinetics. Exosomes can potentially increase circulation time, preserve drug therapeutic activity, increase drug concentration in the target tissue or cell to augment therapeutic efficacy, while simultaneously reducing exposure of healthy tissues to reduce toxicity. Since they are nanosized and carry cell surface molecules, exosomes can cross various biological barriers that might not be possible with free drugs or targeting agents.

One of the concerning factors for determining in vivo distribution in tumor model was enhanced permeability and retention (EPR) effect by which nanoparticles tend to concentrate in tumor tissue much more than they do in normal tissues. Although, only a fraction (0.7% median) of the total administered nanoparticle dose is usually able to reach a solid tumor, which might give false positive signals of exosome distribution. Surprisingly, we did not observe any retention of radioactivity for free ¹¹¹In-oxine, and non-targeted or non-cancerous exosomes (HEK293 exo). This implies that our demonstration of exosome biodistribution and targeted therapy is not an EPR effect, rather the exosomes were directed toward target organs by over-expressed precision peptide on their surface.

Many mechanisms have been implemented to boost the antitumor activities of therapeutic antibodies, including extended half-life, blockade of signaling pathways, activation of apoptosis and effector-cell-mediated cytotoxicity. Here we propose to target Fc gamma-receptor (FcR) based platform to deplete of M2 macrophages. The direct effector functions that result from FcR triggering are phagocytosis, ADCC, and induction of inflammation; also, FcR-mediated processes provide immune-regulation and immunomodulation that augment T-cell immunity and fine-tune immune responses against antigens. With respect to IgG2b, part of the most potent IgG subclasses can bind specifically into FcRIII (KD=1.55×10⁻⁶) and IV (KD=5.9×10⁻⁸) to activate FcRs.^{[35,36]} Peptibodies containing myeloid-derived suppressor cells (MDSC)-specific peptide fused with Fc portion of IgG2b was able to deplete MDSCs in vivo and retard tumor growth of a lymphoma mouse model without affecting proinflammatory immune cells types, such as dendritic cells.^{[37]} This plasticity of effector and immune-regulatory functions offers unique opportunities to apply FcR-based platforms and immunotherapeutic regimens for vaccine delivery and drug targeting against infectious and non-infectious diseases.

### 1. CD206-Positive M2 Macrophage-Targeting Exosomes

Investigators have used tumor cells, dendritic cells (DCs), mesenchymal stem cells (MSCs), MDSCs, endothelial progenitor cells (EPCs), neural stem cells (NSCs), and other cell types to generate engineered and non-engineered exosomes for both imaging and therapeutic purpose. We have also used tumor cells, MDSCs, EPCs, and NSCs derived exosomes in our previous and ongoing studies. Tumor cell-derived exosomes carry antigens and elicit immunogenic reaction, therefore, these exosomes have been used in studies for tumor vaccination. On the other hand, both MSCs and MDSCs derived exosomes have shown to be immune suppressive. EPC-derived exosomes may enhance neovascularization in the tumors. Therefore, using these cells to generate engineered exosomes to carry CD206 targeting peptide may initiate unwanted effect of immune activation, immune suppression, or neovascularization. Moreover, in vitro growth of MSCs, NSCs, and EPCs may be limited due to cell passage number. Ideal cell to generate engineered exosomes should have the following criteria: 1) Non-immunogenic, 2) unlimited cell passage capacity without changing their characteristics, 3) abundant production of exosomes both in normal and strenuous conditions, 4) cells that can easily be genetically modified. HEK 293 cell is ideal for the production of engineered exosomes. These cells have been extensively used by the biotechnology industry to produce FDA (food and Drug Administration) approved therapeutic proteins and viruses for gene therapies. Exosomes derived from these cells show no immune activation or suppression following long-term injections in animal models. We used HEK293 cells to generate our engineered exosomes to carry precision peptide to target CD206+ M2 macrophages.

The data provided in the Examples shows that exosomes targeting M2 macrophages are utilized effectively to diagnose, monitor, and prevent tumor growth and metastasis for better survival.

CD206-positive M2 macrophage-targeting exosomes and methods of use thereof are provided. One embodiment provides a CD206-positive M2 macrophage-targeting exosome expressing a CD206 binding peptide and an Fc portion of IgG2b. In some embodiments, the CD206 binding peptide is encoded by a nucleic acid sequence having 95%, 99%, or 100% sequence identity to SEQ ID NO:2 and the IgG2b is encoded by a sequence having 95%, 99%, or 100% sequence identity to SEQ ID NO:6.

Another embodiment provides a vector encoded by a nucleic acid sequence having 85%, 90%, 95%, or 100% to SEQ ID NO:5. The vector is useful for producing CD206-positive M2 macrophage-targeting exosomes. One embodiment provides a method for making CD206-positive M2 macrophage-targeting exosomes by transfecting HEK293 cells with the vector and culturing them in exosome free growth media and harvesting the CD206-positive M2 macrophage-targeting exosomes from HEK293 conditioned media. The collected exosomes were shown to deplete polarized macrophage cells (RAW264.7) in the presence of IL4 and IL-3In some embodiments the macrophage are RAW264.7 macrophage cells.

In some embodiments, the CD206-positive M2 macrophage-targeting exosomes are loaded with cargo. The cargo is selected from the group consisting of a detectable label, a chemotherapeutic agent, and a cytotoxic agent.

Another embodiment provides a pharmaceutical composition including the disclosed CD206-positive M2 macrophage-targeting exosomes and a pharmaceutically acceptable excipient.

### 2. RVG Neuron Targeting Engineered Exosomes

In one embodiment, the disclosure provides neuron-targeting peptide (rabies virus glycoprotein) based engineered exosomes to deliver Neuroglobin (Ngb) into the neurons.

The blood brain barrier (BBB) is a system-wide membrane barrier that prevents the brain uptake of circulating drugs, protein therapeutics, RNAi drugs, and gene medicines. Drugs or genes can be delivered to the human brain for the treatment of serious brain disease either (a) by injecting the drug or gene directly into the brain, thus bypassing the BBB, or (b) by injecting the drug or gene into the bloodstream so that the drug or gene enters the brain via the transvascular route across the BBB. With intra-cerebral administration of the drug, it is necessary to drill a hole in the head and perform a procedure called craniotomy. In addition to being expensive and highly invasive, this craniotomy based drug delivery to the brain approach is ineffective, because the drug or gene is only delivered to a tiny volume of the brain at the tip of the injection needle. The only way the drug or gene can be distributed widely in the brain is the transvascular route following injection into the bloodstream. However, this latter approach requires the ability to undergo transport across the BBB. The BBB has proven to be a very difficult and stubborn barrier to traverse safely.

Drug delivery is particularly difficult in the CNS and the methods used for systemic delivery such as intravenous hydrodynamic injection (Ge, Q. et al., Proc Natl Acad Sci USA 101, 8676-81 (2004); Miller, G., Science 297, 1116-8 (2002)) and intravenous (IV or iv) injection of siRNA or shRNA vectors complexed with lipofectamine or polyethyleneimine (Schlachetzki, F., et al., Neurology 62, 1275-81 (2004)) are unlikely to work for delivery to the CNS because of the presence of BBB. Thus, the only available method for CNS delivery at present is local sterotaxic injection of nonreplicating viral vectors and siRNA (Hacein-Bey-Abina, S. et al., N Engl J Med 348, 255-6 (2003)). One problem with these approaches is the extremely limited spread, confining delivery to a small area at the site of injection. Thus, delivery methods to ensure a more extensive spread of the delivered si/shRNAs for its efficacy in situations like tumors and intracranial infections or strokes are needed.

Studies have shown that a peptide derived from Rabies virus glycoprotein (RVG) can specifically target neuronal cells (U.S. Pat. No. 9,757,470B2). This peptide has previously been shown to competitively inhibit a-bungarotoxin binding to the nicotinic acetylcholine receptor α7 subunit (Notter, M. F. & Leary, J. F., J Cell Physiol 125, 476-84 (1985); Chen, T. J., et al., Chin J Physiol 48, 129-38 (2005); Gotti, C. & Clementi, F., Prog Neurobiol 74, 363-96 (2004); Plakhov, I. V., et al., Virology 209, 257-62 (1995)). Acetylcholine receptor α7 subunit is widely expressed by many cell types in the brain including the neurons, astrocytes and glia cells and it is also expressed by the brain capillary endothelial cells (Wender, P. A. et al., Proc Natl Acad Sci USA 97, 13003-8 (2000)).

RVG has been successfully used to transport a variety of nanocarriers into the CNS, such as liposomes, polymers, dendrimers, exosomes, micelles, bioactive proteins, and moieties with efficiency and fast permeability (Wang, Q. et al., RSC advances vol. 11,15 8505-8515 (2021)). Also, when RVG-carriers provide biological macromolecules to the CNS, RVG modified carriers exhibit high load capacity.

Receptor-mediated endocytosis is the mechanism for RVG-carrier to penetrate through the BBB modified with RVG nanocarriers and primarily bind to nAChR or γ-aminobutyric acid (GABA) receptor, which is widely located on the extracellular surface of target cells, such as microvascular endothelial cells and neurons (Wang, Q. et al., RSC advances vol. 11, 15 8505-8515 (2021)). RVG specifically recognizes nAChR or GABA-R) to initiate transcytosis processes to deliver carriers into CNS in a noninvasive way.

### 3. Myeloid Cell Targeting Engineered Exosomes

In one embodiment, the disclosure provides myeloid cell targeting peptide engineered exosomes that targets and kills MDSC by antibody-dependent cell-mediated cytotoxicity (ADCC).

a. Myeloid-Derived Suppressor Cells (MDSC)

In another embodiment, the disclosure provides myeloid-derived suppressor cells (MDSC) targeting peptide engineered exosome that targets and kills MDSC by ADCC.

Myeloid-derived suppressor cells (MDSC) are a group of heterogeneous cells of immature hematopoietic myeloid-cell progenitors, exerting immunosuppressive functions and therefore negatively regulating immune responses (Gabrilovich, D. I., and Nagaraj, S, Nat. Rev. Immunol. 9, 162-174 (2009); Talmadge, J. E., and Gabrilovich, D., Nat. Rev. Cancer 13,739-752(2013)). MDSC were found to be accumulated in many pathological conditions and to exploit a plethora of redundant mechanisms to influence both innate and adaptive immune responses (Dilek, N., et al., Curr Opin Organ Transplant 15, 765-768(2010); Gabrilovich, D. I., et al., Nat. Rev. Immunol. 12, 253-268 (2012)). MDSC are potent inhibitors of T and B lymphocytes activation, proliferation and responses, in particular by nutrients (e.g. L-arginine and L-cysteine) depletion mechanisms, the production of reactive oxygen species (ROS) and reactive nitrogen species (RNS), perturbation of T-lymphocyte trafficking (e.g. L-selectin expression decrease and aberrant chemokine release), induction of apoptosis (via Galectin 9) and by deviating T-lymphocytes differentiation towards Th-17 responses through IL-1β production (Bruchard et al., Nat. Med. 2013). MDSC have also the extraordinary capacity to expand antigen-specific natural regulatory T cells (nTreg), to promote conversion of naive T cells into induced Treg (iTreg) cells and to promote Treg infiltration at inflamed, infected or tumor sites. MDSC were also described to decrease the number and inhibit function of NK cells, in particular by membrane bound TGFβ. Furthermore, in analogy with the immune deviation they induce in T cells responses, MDSC skew macrophages towards an M2 phenotype (non-inflammatory macrophages) by inhibiting macrophages production of IL-12. Similarly, MDSC impair dendritic cell (DC) function by producing IL-10, which also inhibits IL-12 production by DC and reduces DC capacity to activate T cells. Finally, MDSC act on non-hematopoietic cell and have been in particular widely recognized to facilitate tumor angiogenesis, tumor spread, tumor-cell invasion and metastasis (Keskinov, A. A., and Shurin, M. R., Immunobiology (2014); Ye, X.-Z, et al., J Genet Genomics 37,423-430 (2010)).

b. Colony Stimulating Factor 1 Receptor (CSF1R)

In another embodiment, the disclosure provides a CSF1R targeting peptide engineered exosome that targets and kills myeloid cells by antibody-dependent cell-mediated cytotoxicity (ADCC).

Colony stimulating factor 1 receptor (referred to herein as CSF1R; also referred to in the art as FMS, FIM2, C-FMS, and CD115) is a single-pass transmembrane receptor with an N-terminal extracellular domain (ECD) and a C-terminal intracellular domain with tyrosine kinase activity. Ligand binding of CSF1 or the interleukin 34 ligand (referred to herein as IL34; Lin et al., Science 320: 807-11 (2008)) to CSF1R leads to receptor dimerization, upregulation of CSF1R protein tyrosine kinase activity, phosphorylation of CSF1R tyrosine residues, and downstream signaling events. Both CSF1 and IL34 stimulate monocyte survival, proliferation, and differentiation into macrophages.

Many tumor cells have been found to secrete CSF1, which activates monocyte/macrophage cells through CSF1R. The level of CSF1 in tumors has been shown to correlate with the level of tumor-associated macrophages (TAMs) in the tumor. Higher levels of TAMs have been found to correlate with poorer patient prognoses. In addition, CSF1 has been found to promote tumor growth and progression to metastasis in, for example, human breast cancer xenografts in mice. See, e.g., Paulus et al., Cancer Res. 66: 4349-56 (2006). Further, CSF1R appears to play a role in osteolytic bone destruction in bone metastasis, as a small molecule inhibitor of receptor tyrosine kinase activity suppresses that destruction. See, e.g., Ohno et al., Mol. Cancer Ther. 5: 2634-43 (2006).

CSF1 and its receptor have also been found to be involved in various inflammatory and autoimmune diseases. See, e.g., Hamilton, Nat. Rev. 8: 533-44 (2008). For example, synovial endothelial cells from joints afflicted with rheumatoid arthritis have been found to produce CSF1, suggesting a role for CSF1 and its receptor in the disease. Blocking CSF1R activity with an antibody results in positive clinical effects in mouse models of arthritis, including a reduction in the destruction of bone and cartilage and a reduction in macrophage numbers. See, e.g., Kitaura et al., J. Clin. Invest. 115: 3418-3427 (2005).

Mature differentiated myeloid lineage cells such as macrophages, microglial cells, and osteoclasts contribute to pathology of various diseases such as rheumatoid arthritis, multiple sclerosis and diseases of bone loss. Differentiated myeloid lineage cells are derived from peripheral blood monocyte intermediates. CSF1R stimulation contributes to development of monocytes from bone marrow precursors, to monocyte proliferation and survival, and to differentiation of peripheral blood monocytes into differentiated myeloid lineage cells such as macrophages, microglial cells, and osteoclasts. CSF1R stimulation thus contributes to proliferation, survival, activation, and maturation of differentiated myeloid lineage cells, and in the pathologic setting, CSF1R stimulation contributes to the ability of differentiated myeloid lineage cells to mediate disease pathology.

Targeting CSF1R signaling would therefore be useful in the treatment of various CSF1R-related diseases, such as cancer, inflammatory conditions, arthritis, and autoimmune diseases.

c. Antibody Dependent Cellular Cytotoxicity

The term “antibody dependent cellular cytotoxicity” or “ADCC” refers to a cell mediated reaction in which non-specific cytotoxic cells (e.g. NK cells, neutrophils, macrophages, etc.) recognize antibody bound on a target cell and subsequently cause lysis of the target cell. Such cytotoxic cells that mediate ADCC generally express Fc receptors (FcR). The primary cells for mediating ADCC (NK cells) express FcγRIII, whereas monocytes express FcγRI, FcγRII, FcγRIII, and/or FcγRIV. To assess ADCC activity of a molecule, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model.

### 4. GMB Targeting Engineered Exosomes

In another embodiment, the disclosure provides a glioblastoma multiforme (GBM) targeting peptide engineered exosome that targets and kills glioma cells by antibody-dependent cell-mediated cytotoxicity (ADCC).

a. Glioblastoma Multiforme

Gliomas are the most common brain tumors, and, in particular, grade IV glioma, also named glioblastoma multiforme (GBM), represents the most severe type. This highly aggressive tumor develops either de novo (primary GBM) or as consequence of the malignant progression from low-grade glioma (secondary GBM). In both cases, due to the intrinsic characteristic of this tumor, prognosis is poor and the median survival when radiotherapy and chemotherapy are combined is 14.6 months (12.).

Glioblastoma multiforme is a highly heterogeneous tumor, which shows clear cellular and tissue dissimilarities, displaying a strong hemorrhagic component and wide areas of necrosis, which, at the microscopic level, coincide with the presence of massive microvascular proliferation and pseudopalisading patterns (9).

Most importantly, GBM is characterized by a diffuse tissue-distribution pattern, with extensive dissemination of the tumor cells within the brain that hinders complete surgical resection. Therefore, disease recurrence occurs in the majority of the patients. Moreover, GBM progression is also accompanied by extensive neovascularization.

The need and importance is increasingly felt for the development of new markers, and for the identification of specific molecular targets for the development of a therapy for blocking the growth and diffusion of GBM and for its treatment.

In the last decade, several seminal studies have dealt with the identification and isolation of cancer stem cells (CSCs), which might be responsible for the initiation, maintenance and progression of different types of tumors. It has been suggested that the persistent growth of cancers, clonal diversification and evolution, tumor metastasis and recurrence after therapy may be the consequence of the maintenance by tumor cells of the proliferative potential of stem cells from which the tumor may have originated, which are called cancer stem cells. Transformed stem-like cells have been found in hematopoietic malignancies, breast cancer and stem-like neural progenitors also in human brain tissues.

b. Eph (Erythropoietin-Producing Hepatocellular Carcinoma) Receptors

The Eph (erythropoietin-producing hepatocellular carcinoma) receptors comprise the largest family of tyrosine kinases encoded in the human genome and play important roles in development and disease receiving an external stimulus and responding by transmitting a signal to the inside of the cell, starting numerous processes that are vital for the maintenance of organism function. They can be distinguished from other RTKs in that they all recognize ligands, known as ephrins, which are anchored to the membrane of apposing cells. Ligand binding typically triggers tyrosine phosphorylation of Eph receptors.

Within key molecules in GBM development are the Eph receptors family members. The Eph receptors are mainly expressed in early development and are crucial for embryonic development, regulating processes such as cell migration and adhesion (Liu D. P. et al., Int. J. Oncol. 2007; 30: 865-871). Expression of Eph receptors that is very low in adult and differentiated tissues becomes upregulated in a number of human malignancies such as melanoma and breast, lung, and ovarian cancer, as well as glioma. In these malignancies, contrary to an Eph receptor, the endogenous ligand ephrin appears downregulated. Eph receptor A2 (EphA2) has been proposed as a novel molecular marker and therapeutic target since it is strongly overexpressed in GBM cells but not in normal brain (Puttick S. et al., Mol. Imaging. 2015; 14: 385-399; Wykosky J., et al., Mol. Cancer Res. 2005; 3: 541-551). The overexpression of EphA2 correlates with poor patient outcome, and it is essential in the maintenance of the pool of GSCs, promoting their invasiveness in vivo. EphA2 overexpression promotes GSC tumorigenesis in GBM, and its blockage strongly induces a tumor-suppressive phenotype. Moreover, EphA2 is co-expressed with other stem cell markers such as CD133 and integrin alpha 7 (Affinito., A. et al., Molecular Therapy Nucleic Acids, 20:176-185 (2020)).

### 5. Neutrophil Targeting Engineered Exosomes

In another embodiment, the disclosure provides a neutrophil targeting peptide engineered exosome that targets and kills cells by antibody-dependent cell-mediated cytotoxicity (ADCC).

After an ischemic stroke, neutrophils are among the first cells in the blood to respond, contributing to disruption of the blood brain barrier (BBB), cerebral edema, and brain injury (Segel GB, et al., J Leukoc Biol 2011; 89: 359-372. The response is mediated by factors released from neutrophils including reactive oxygen species (ROS) (superoxide, hypochlorous acid), proteases (matrix metalloproteinases, elastase, cathepsin G, proteinase 3), cytokines (IL-1β, IL-6, IL-8, tumor necrosis factor alpha (TNF-α)), and chemokines (CCL2, CCL3, CCL5) (Kolaczkowska E, Kubes P. Nat Rev Immunol 2013; 13: 159-175.Neutrophils also are involved in thrombosis and atherosclerosis, which are known to be major processes that cause ischemic stroke (Jickling, Glen C et al. Journal of cerebral blood flow and metabolism: official journal of the International Society of Cerebral Blood Flow and Metabolism vol. 35,6 (2015): 888-901). They promote clot formation through interactions with platelets, proteolytic cleavage of clotting factors (tissue factor pathway inhibitor (TFPI) and coagulation factor X), and release of prothrombotic molecules (neutrophil extracellular traps (NETs) and tissue factor). Neutrophils promote atherosclerosis and plaque rupture by enhancing monocyte infiltration, producing oxidized low density lipoprotein (oxLDL), and releasing proteolytic enzymes that degrade the fibrous cap.

Neutrophils are also known to cause exacerbation of stroke due to accumulation of activated neutrophils (peaked at 48 hrs) at the stroke site causing leakage of blood brain barrier (BBB) and extravasation of fluid and protein. Depleting neutrophils using engineered exosomes decreased stroke volume as well as BBB leakage.

Due to the involvement of neutrophils in ischemic stroke, they have emerged as treatment targets to reduce infarct size and improve stroke outcomes

### EXAMPLES

### Methods and Materials

### Cell Lines

4T1, a murine mammary carcinoma cell line from a BALB/cfC3H mouse, was originally obtained from the American Type Tissue Culture Collection (ATCC), and modified by Dr. Hasan Korkaya (Augusta University) to express the luciferase gene reporter. For cell cultures and propagation, both cells were grown in Roswell Park Memorial Institute 1640 medium (RPMI) (Thermo Scientific), supplemented with 10% fetal bovine serum (FBS) (Nalgene-GIBCO), 2 mM glutamine (GIBCO, Grand Island, NY, USA) and 100U/mL penicillin and streptomycin (GIBCO, Grand Island, NY, USA) at 5% CO₂ at 37° C. in a humidified incubator. For the generation of exosomes, cells (5×10⁶ cells in T175 flask) were grown in RPMI-1640 media containing 10% exosome free FBS and incubated in a humidified incubator in hypoxic condition (1% oxygen) for 48 hours. Mouse Embryonic Fibroblast cell line (MEF) was obtained from Dr. Nahid Mivechi's laboratory and both cell lines and Human embryonic kidney 293 cell line (HEK293) was obtained from Dr. Satyanarayana Ande of Augusta University were grown in Dulbecco's Modified Eagle Medium (DMEM) (Corning, NY, USA) containing 10% exosome free PBS. HEK293 cells were transfected with lentivirus to develop engineered exosomes. RAW264.7 mouse macrophage cell line was obtained from Dr. Gabor Csanyi in the vascular biology department at Augusta University and used for in vitro targeting and cytotoxicity assays. RAW264.7 were grown in DMEM media containing 10% FBS.

### Exosome Isolation

Exosomes were isolated from the culture supernatants of 4T1, HEK293 cells and transfected HEK293 cells. Briefly, 5×10⁶ cells were plated in 175 cm2 flasks and grown overnight with 10% FBS complete media in normoxia (20% oxygen). The media was removed and replenished with exosome-free complete media. Exosomes were depleted from the complete media by ultracentrifugation for 70 minutes at 100,000×g using an ultracentrifuge (Beckman Coulter) and SW28 swinging-bucket rotor. The cells were then grown for 48 hours under normoxic condition. The cell culture supernatant was centrifuged at 700×g for 15 minutes to get rid of cell debris. To isolate exosomes, we employed combination of two steps of size-based method by passing through 0.20 μm syringe filter and centrifugation with 100 k membrane tube at 3200×g for 30 minutes followed by a single step of ultracentrifugation at 100,000×g for 70 minutes (as described in our previous publication.²⁶

### Nanoparticle Tracking Analysis

Nanoparticle tracking analysis (NTA) was performed using ZetaView, a second-generation particle size instrument from Particle Metrix for individual exosome particle tracking as described previously²⁶. This is a high performance integrated instrument equipped with a cell channel, which is integrated into a ‘slide-in’ cassette and a 405-nm laser. Samples were diluted in 1×PBS between 1:100 and 1:2000 and injected in the sample chamber with sterile syringes (BD Discardit II, New Jersey, USA). All measurements were performed at 23° C. and pH 7.4. As measurement mode, we used 11 positions with 2 cycles, and for analysis parameter, we used maximum pixel 200 and minimum 5. ZetaView 8.02.31 software and Camera 0.703 μm/px were used for capturing and analyzing the data.

### Flow Cytometry

The common exosome markers, mouse-specific anti-CD9 FITC, and anti-CD63 APC antibody (Biolegend, San Diego, CA, USA) were used to label exosomes at 4° C. for 30 minutes. Flow cytometry samples were acquired using Accuri C6 flow cytometer (BD Biosciences) with the threshold set at 10 and analyzed by BD Accuri C6 software. For in vivo flow cytometric analysis, the fresh tissue collected was disseminated into single cells, filtered through a 70 μm cell strainer, and spun at 1,200 rpm for 15 minutes. The pellet was re-suspended in 1% BSA/PBS, and incubated with LEAF blocker in 100 μL volume for 15 minutes on ice to reduce non-specific staining. The single cells were then labeled to detect the macrophage and immune cell populations using fluorescence conjugated antibodies such as CD3, CD4, CD8, CD206, F4/80 and IgG2b. All antibodies were mouse specific and the samples were acquired using Accuri C6 flow cytometer (BD Biosciences).

### Tumor Model

4T1 cells expressing the luciferase gene were orthotopically implanted in syngeneic BALB/c (Jackson Laboratory, Main USA). All the mice were between 5-6 weeks of age and weighing 18-20 g. Animals were anesthetized using a mixture of Xylazine (20 mg/Kg) and Ketamine (100 mg/Kg) administered intraperitoneally. Hair was removed for the right half of the abdomen by using hair removal ointment, and then abdomen was cleaned by Povidone-iodine and alcohol. A small incision was made in the middle of the abdomen, and the skin was separated from the peritoneum using blunt forceps. Separated skin was pulled to the right side to expose the mammary fat pad and 50,000 4T1 cells in 50 μL Matrigel (Corning, NY, USA) were injected. Tumor growth was monitored every week. In vivo, optical images were obtained every week to keep track of primary tumor and metastasis development by injecting 100 μL of luciferin (dose 150 mg/kg) intraperitoneally followed by the acquisition of bioluminescence signal by spectral AmiX optical imaging system (Spectral instruments imaging, Inc. Tucson, AZ). The photon intensity/mm/sec was determined by Aura imaging software by Spectral Instruments Imaging, LLC (version 2.2.1.1). The animals were anesthetized using an isoflurane vaporizer chamber (2.5% Iso: 2±3 L/min 02) and maintained under anesthesia (2% with oxygen) during the procedure.

### Radiolabeling of Exosomes Using Indium-111 (111In)

Exosomes were labeled with In-111-oxine using our optimized method of labeling²³. In brief, exosomes (fresh or thawed) were washed with normal saline, reconstituted at 12 billion exosomes/ml, incubated with 1 mCi of In-111-oxine in normal saline for 30 minutes at room temperature. Then free from bound In-111 will be separated using Amicon ultra centrifugal filters with a cut off value of 100 kDa for 30 minutes at 3200×g at 20° C. Serum challenge studies were used to determine any dissociation over 24 hours, which was determined by thin-layered paper chromatography (TLPC).

### Thin Layer Paper Chromatography for Radiolabeling Efficacy and Stability

3MM Whatman® cellulose chromatography paper was cut into 1×8 cm small pieces. The bottom spotted point was made by 5 μL of each sample followed by submerging the bottom part of each piece (fluid level remained below the spotted point) into the eluent consisting of 100% methanol and 2M Sodium acetate solution (1:1). Then the pieces were allowed to remain upright until the eluent reaches the top part. The pieces were cut into the top and bottom halves and were subsequently put in the glass tubes for the measurement of emitted gamma activity by Perkin-Elmer Packard Cobra II Auto-Gamma Total radioactivity was calculated by combining the activity from top and bottom halves. To determine the percent dissociation of bound ¹¹¹In-oxine from exosomes, labeled exosomes were challenged with serum at 37° C. up to 24 hrs or 48hrs. At different time points, free ¹¹¹In-oxine, and serum challenged labeled exosomes were tested using thin layer paper chromatography as described above to determine the percent of bound vs. free ¹¹¹In-oxine.

### In Vivo SPECT/CT Imaging of 111In-Oxine-Labeled Exosomes

After the intravenous injection of 350±50 μCi of 111In-oxine-labeled exosomes in 100 μL into the tail vein of the mice, whole body SPECT images were acquired using our previously published protocol with a dedicated 4-headed NanoScan, high-sensitivity microSPECT/CT 4R (Mediso, Boston, MA, USA) fitted with high-resolution multi-pinhole (total 100) collimators. The microSPECT has a wide range of energy capabilities from 20 to 600 keV, with a spatial resolution of 275 μm. The images were obtained using 60 projection images with 60 seconds/projection, with a medium field of view. Attenuation was corrected using concurrent computed tomographic (CT) images, and then the images were reconstructed with low iteration and low filtered back-projection. The image acquisitions were commenced 3 hours after the injection of ¹¹¹In-oxine-labeled exosomes. During the whole procedure, the animals were anesthetized and maintained using a combination of 1.5% isoflurane and 1 L/min medical oxygen flow and their body was immobilized in an imaging chamber to restrain movements. Throughout the scanning their body temperature was maintained at 37° C. and breathing was monitored.

### Quantitative Analysis of Radioactivity in Individual Organ

Reconstructed analyze formatted file was used in ImageJ (Wayne Rasband, National Institutes of Health, USA) version 1.51a for both CT and SPECT analysis. The primary tumor, a metastatic site in the lungs and other organs were identified by orthogonal, dorsal and ventral views from the resliced stack images. Z stack images were created from the CT and SPECT of the individual organ for depth and anatomical accuracy of the organ. Total radioactivity was determined by the sum of the values of the pixels (RawIntDen) in the selected region of interest (ROIs) around the organs. The activity in the individual organ was expressed in percent of activity in the whole body (total radioactivity dose).

### Ex Vivo Quantification of Gamma Activity of Individual Organ

After the final scan, animals were euthanized, and their organs were harvested and weighed. Emitted gamma radiation from each organ was measured by Perkin-Elmer Packard Cobra II Auto-Gamma after transferring them into the individual glass tube.

### Determination of Specificity of Precision Peptide In Vitro and In Vivo

Biotinylated precision peptide (Biotin-CSPGAKVRC) (SEQ ID NO:1) was custom synthesized by a commercial vendor (GeneScript, Piscataway, NJ) using standard peptide synthesis and biotin was attached to the N-terminus. For both in vitro and in vivo studies, biotinylated peptide was labeled with rhodamine using rhodamine-tagged streptavidin utilizing standard protocol for labeling supplied by the vendor (ThermoFisher Scientific). Rhodamine-labeled peptide was used in in vitro studies to determine the specific uptake to CD206 sites on RAW 264.7 cells with or without blocking CD206 receptor using a CD206 blocking peptide (Cat #MBS823969, Mybiosource). All cells were pre-incubated with anti-CD44 antibody to block non-specific phagocytosis. All cells were stained for CD206 (fluorescein, FITC) and counter stained with DAPI.

For in vivo specificity, rhodamine labeled peptide (red) was injected intravenously (IV) in metastatic syngeneic murine breast cancer (4T1) bearing Balb/C mice. Three hours after IV administration, all animals were euthanized, and lungs, spleen and tumors were collected for immunohistochemical analysis. Frozen sections from the collected tissues were stained for CD206 (fluorescein, FITC) and counter stained with DAPI.

### Labeling of Conjugated-Precision Peptide With Tc99m:

Hydrazine Nicotinamide (HYNIC)-conjugated M2-targeting precision peptide was custom synthesized by a commercial vendor (GeneScript, Piscataway, NJ) using standard peptide synthesis. Then, 250 μg of HYNIC-M2-targeting conjugated peptide was radio labeled with 99m-Tc-pertechnetate in the presence of a solution containing tricine (14.4 mg/mL—Acros organics) and stannous chloride (0.5 mg/mL—Acros organics) in oxygen free condition (air was purged by N2). Following this step, we centrifuged the mixture to remove the unconjugated peptide using 1K centrifugal filter at 3200×g for 15 min. The amount of radiolabeled peptide was detected using a dose calibrator (CRC-25R—Capintec, Inc.). A dose of approximately 300 μCi of radiolabeled peptide was injected per animal.

### Construction for Overexpressing CD206+ M2-Macrophage Targeting Peptide and Fc Portion of Mouse IgG2b on the Exosome Surface:

Two different lentiviral vector constructs were made by 3rd party vendor (VectorBuilder Inc, TX, USA) and used to generate engineered exosomes in HEK293 cells. CD206+ M2-macrophage targeting peptide and Fc portion of mouse IgG2b along with mouse LAMP2b protein were custom designed and inserted into third-generation lentivirus vector (eBiosciences). QIAquick Gel Extraction Kit (Qiagen, Valencia, CA, USA) and Plasmid Midi Kit (Qiagen, Valencia, CA, USA) were used to extracting the plasmid DNA.

### Biogenesis of Engineered Exosomes Expressing Precision Peptide and Fusion Protein

For the lentiviral production, 1×10⁶ HEK293TN cells were seeded in a 100 mm culture dish. At 70-75% of confluency, after removing the old media, the cells were supplemented with lentivirus producing plasmids and the targeting cloning plasmid in the presence of Opti-mem and Lipofectamine2000. After 24 hours, the culture supernatants containing virus particles were collected, followed by centrifugation and filtration through 0.45 μm PVDF membrane to get rid of the cell debris. For the transfection using lentivector, 500,000 HEK293 cells were seeded in a 100 mm culture dish. At 70-75% of confluency, after removing the old media, the cells were supplemented with a transfection cocktail containing regular media, lentivirus, and polybrene. The cells were expanded and subsequently selected with 300 μg/mL neomycin for 4 weeks. The transfection of selected cells was confirmed by luciferase activity of the cells following the addition of luciferin. After collecting the supernatant from 6×10⁶ transfected HEK293 cell cultures incubated for 48 hours in a T175 flask with exosomes free media, the supernatant was centrifuged at 700×g for 15 minutes to remove cell debris. Then it was filtered through a 0.20 μm PVDF (low protein attachment) membrane and centrifuged using Amicon ultra centrifugal filters with a cut off value of 100 kDa for 30 minutes at 3200×g followed by a final washing step with ultracentrifugation at 100,000×g for 70 minutes.

Labeling of Exosomes with Dil

Dil-labeled exosomes were used to demonstrate targeting efficiency of the engineered exosomes both in vitro and in vivo. Following isolation, exosomes were re-suspended in 1 mL of Dil working solution (final concentration 5 μM/mL in PBS). After 30 minutes of incubation at 37° C., free Dil was removed by two centrifugation wash steps with PBS using 100k membrane tubes.

### Immunofluorescent Staining of Adherent Cell Cultures

18×18-1 glass coverslips were soaked in 100% ethanol for sterilization followed by washing in PBS and then each of them was transferred to each well of 6 well-plates. 300,000 RAW264.7 cells were seeded and incubated overnight. Then the adherent cells were treated with Dil-labeled exosomes (20 μL containing approximately 3×10⁸ exosomes) and incubated for 4-6 hours. After that, media with exosomes was removed and the cells were rinsed twice with PBS. Cells were fixed with 3% paraformaldehyde for 15 minutes followed by washing with PBS. Cells were covered with blocking solution and incubated for 20-30 minutes at room temperature. Blocking solution was gently flicked away and appropriate antibody (Alexa 488 anti-mouse CD206 antibody) diluted in blocking solution (1:100) was added. After 2 hours of incubation the antibody was removed and the cells were washed with PBS followed by counter staining with DAPI for nuclear stain. After final wash step, the coverslips were transferred for mounting on slides using ProLong™ Gold Antifade mounting media (Invitrogen™).

### Determination of Specificity of Engineered Exosomes In Vitro and in Vivo

In vitro studies: Raw264.7 (CD206+ cells) and mouse embryonic fibroblasts (MEF, CD206− cells) were used as model cells for in vitro studies of CD206 specificity for engineered exosomes. The anti-CD44 antibody was used before adding the exosomes to block the non-specific uptake of added exosomes by the process of phagocytosis. Both Raw264.7 and MEF cells, grown in small tissue culture petri-dish, were treated with anti-CD44 antibody to block phagocytosis, and then these cells were incubated with fluorescent dye Dil labeled engineered and control exosomes collected from HEK293 cells with or without CD206 blocking peptide (Cat #MBS823969, Mybiosource). CD206 blocking peptide was used to determine the specificity of the engineered exosomes expressing precision CD206 targeting peptide to target CD206 sites. Cells were stained with an anti-CD206 antibody plus FITC tagged secondary antibody. High-resolution fluorescent microscopy images were obtained.

### In Vivo Studies Using Dil Labeled Exosomes

For in vivo specificity studies, Balb/c mice bearing 4T1 tumors were used, which were treated with either vehicle or anionic clodronate liposome (Clophosome®-A) 24 hours before the administration of control or engineered exosomes. Clophosome®-A is composed of anionic lipids which deplete more than 90% macrophages in the spleen after a single intravenous injection^{24,25}. Clophosome®-A is not approved for human studies, and it was used for experimental use only. Orthotopic breast cancer was developed by injecting 50,000 cells in the fat pad of right lower breast. Untreated animals were used as a positive control, and Clophosome®-A treated animal were used as negative control. 24 hours after the treatment (5 weeks old tumor-bearing animals), the mice were used to determine the accumulation of IV administered Dil labeled control and engineered exosomes in the tumors, spleen, liver and lungs. Three hours after IV administration of exosomes the organs were harvested with proper perfusion. Half of the tumors and organs including lymph nodes were fixed, and sectioned for immunohistochemical studies. Immunohistochemistry was conducted to determine the accumulation of Dil labeled exosomes in CD206+ and CD206− cells.

### Immunofluorescent Staining of Frozen Sections

Harvested tissues (tumor, spleen and Lungs) from the animals were transferred to 30% sucrose and 3% paraformaldehyde solution. 10 μm thick sections were prepared and collected on to pre-warmed slides, and allowed to dry at least for a day. Sections were covered with ˜200-μL of blocking solution and were placed in the humidity box for 20-30 minutes at room temperature. Blocking solution was gently flicked away and appropriate primary antibodies diluted in blocking solution was added. The slides were incubated in humidity box overnight at 4° C. Then the slides were washed twice at least 5 minutes per wash. Secondary antibodies diluted in blocking solution was added to the sections and incubated at room temperature for two hours in humidity box or overnight at 4° C. Then the slides were washed twice at least 5 minutes per wash followed by counter stain with DAPI for nuclear stain. After final wash step, slides were mounted with ProLong™ Gold Antifade mounting media (Invitrogen™) and with an 18×18-1 glass coverslips.

### Western Blot

Cells and tissues were processed for protein isolation using Pierce RIPA buffer (Thermo Scientific, USA). Protein concentrations were estimated with Pierce, BCA protein assay kit (Thermo Scientific, USA), and separated by standard Tris/Glycine/SDS gel electrophoresis. Membranes were blocked with Odyssey Blocking buffer (LI-COR, Lincoln, NE) for 60 min at room temperature and incubated with primary antibody against 6×His-tag (SEQ ID NO:7) (BioLegend, cat #362602, 1:500) antibody followed by horseradish peroxidase-conjugated secondary antibody (1:5000,). The blot was developed using a Pierce Super Signal West Pico Chemiluminescent substrate kit (Thermo Scientific, USA). Western blot images were acquired by Las-3000 imaging machine (Fuji Film, Japan).

### Use of Engineered Exosomes Carrying Fusion Protein as Therapeutic Probes

In vitro studies were performed to assess phagocytosis and cytotoxicity using exosome-Fc-mIgG2b complex: CFSE-stained Raw264.7 were used converted to M2 macrophages using IL4 and IL-13 and MEF co-cultured with splenocytes at different ratios. Twenty-four hours after co-culture, engineered exosomes carrying Fc-mIgG2b were added to the co-culture, and the. The studies were repeated at least three times for reproducibility and there was multiple replicate at each time.

### Statistical Analysis

Quantitative data were expressed as mean±standard error of the mean (SEM) unless otherwise stated, and statistical differences between more than two groups were determined by analysis of variance (ANOVA) followed by multiple comparisons using Tukey's multiple comparisons test. Comparison between 2 samples was performed by Student t test. GraphPad Prism version 8.2.1 for Windows (GraphPad Software, Inc., San Diego, CA) was used to perform the statistical analysis. We used a significance level of 5% (α=0.05) and for a power of 80% (the chance of detecting a significant difference if there's any), the sample size required for the experiments were between 3 or 4 animals per group. The same sample size also was valid for a 90% power calculation. For this reason, we fixed our sample size to n=3 or n=4 as mentioned in the methodology. Differences with p-values less than 0.05 were considered significant (*p<0.05, **p<0.01, ***p<0.001, ****p<0.0001).

Plasmid Sequence
Plasmid final version (targeting + therapeutic)
Lamp2b signal peptide-linker-CD206 target
(TGCTCTCCGGGGGCGAAAGTCAGGTGC (SEQ ID NO: 2))-linker-mIgG2b-linker-
Lamp2b c-terminus sequence-6His-tag-stop codon-
(SEQ ID NO: 3)
ATGTGCCTCTCTCCGGTTAAAGGCGCAAAGCTCATCCTGATCTTTCTGTTC
CTAGGAGCCGTTCAGTCCAATGCAGCGCGATGCTCTCCGGGGGCGAAAGT
CAGGTGCGCTCGTGGGCCCATTTCAACAATCAACCCCTGTCCTCCATGCAA
GGAGTGTCACAAATGCCCAGCTCCTAACCTCGAGGGTGGACCATCCGTCT
TCATCTTCCCTCCAAATATCAAGGATGTACTCATGATCTCCCTGACACCCA
AGGTCACGTGTGTGGTGGTGGATGTGAGCGAGGATGACCCAGACGTCCAG
ATCAGCTGGTTTGTGAACAACGTGGAAGTACACACAGCTCAGACACAAAC
CCATAGAGAGGATTACAACAGTACTATCCGGGTGGTCAGCACCCTCCCCA
TCCAGCACCAGGACTGGATGAGTGGCAAGGAGTTCAAATGCAAGGTCAAC
AACAAAGACCTCCCATCACCCATCGAGAGAACCATCTCAAAAATTAAAGG
GCTAGTCAGAGCTCCACAAGTATACATCTTGCCGCCACCAGCAGAGCAGT
TGTCCAGGAAAGATGTCAGTCTCACTTGCCTGGTCGTGGGCTTCAACCCTG
GAGACATCAGTGTGGAGTGGACCAGCAATGGGCATACAGAGGAGAACTA
CAAGGACACCGCACCAGTCCTGGACTCTGACGGTTCTTACTTCATATACAG
CAAGCTCGATATAAAAACAAGCAAGTGGGAGAAAACAGATTCCTTCTCAT
GCAACGTGAGACACGAGGGTCTGAAAAATTACTACCTGAAGAAGACCATC
TCCCGGTCTCCGGGTAGCGGAGGCTTGATAGTTAATTTGACAGATTCAAA
GGGTACTTGCCTTTATGCAGAATGGGAGATGAATTTCACAATAACATATG
AAACTACAAACCAAACCAATAAAACTATAACCATTGCAGTACCTGACAAG
GCGACACACGATGGAAGCAGTTGTGGGGATGACCGGAATAGTGCCAAAA
TAATGATACAATTTGGATTCGCTGTCTCTTGGGCTGTGAATTTTACCAAGG
AAGCATCTCATTATTCAATTCATGACATCGTGCTTTCCTACAACACTAGTG
ATAGCACAGTATTTCCTGGTGCTGTAGCTAAAGGAGTTCATACTGTTAAAA
ATCCTGAGAATTTCAAAGTTCCATTGGATGTCATCTTTAAGTGCAATAGTG
TTTTAACTTACAACCTGACTCCTGTCGTTCAGAAATATTGGGGTATTCACC
TGCAAGCTTTTGTCCAAAATGGTACAGTGAGTAAAAATGAACAAGTGTGT
GAAGAAGACCAAACTCCCACCACTGTGGCACCCATCATTCACACCACTGC
CCCGTCGACTACAACTACACTCACTCCAACTTCAACACCCACTCCAACTCC
AACTCCAACTCCAACCGTTGGAAACTACAGCATTAGAAATGGCAATACTA
CCTGTCTGCTGGCTACCATGGGGCTGCAGCTGAACATCACTGAGGAGAAG
GTGCCTTTCATTTTTAACATCAACCCTGCCACAACCAACTTCACCGGCAGC
TGTCAACCTCAAAGTGCTCAACTTAGGCTGAACAACAGCCAAATTAAGTA
TCTTGACTTTATCTTTGCTGTGAAAAATGAAAAACGGTTCTATCTGAAGGA
AGTGAATGTCTACATGTATTTGGCTAATGGCTCAGCTTTCAACATTTCCAA
CAAGAACCTTAGCTTCTGGGATGCCCCTCTGGGAAGTTCTTATATGTGCAA
CAAAGAGCAGGTGCTTTCTGTGTCTAGAGCGTTTCAGATCAACACCTTTAA
CCTAAAGGTGCAACCTTTTAATGTGACAAAAGGACAGTATTCTACAGCTG
AGGAATGTGCTGCTGACTCTGACCTCAACTTTCTTATTCCTGTTGCAGTGG
GTGTGGCCTTGGGCTTCCTTATAATTGCTGTGTTTATATCTTACATGATTGG
AAGACGGAAAAGTCGTACTGGTTATCAGTCTGTCCACCACCACCACCACC
ACTAA.
Lamp2b signal peptide-
(SEQ ID NO: 4)
ATGTGCCTCTCTCCGGTTAAAGGCGCAAAGCTCATCCTGATCTTTCTGTTC
CTAGGAGCCGTTCAGTCCAATGCA.
Lamp2b c-terminus sequence
(SEQ ID NO: 5)
TTGATAGTTAATTTGACAGATTCAAAGGGTACTTGCCTTTATGCAGAATGG
GAGATGAATTTCACAATAACATATGAAACTACAAACCAAACCAATAAAAC
TATAACCATTGCAGTACCTGACAAGGCGACACACGATGGAAGCAGTTGTG
GGGATGACCGGAATAGTGCCAAAATAATGATACAATTTGGATTCGCTGTC
TCTTGGGCTGTGAATTTTACCAAGGAAGCATCTCATTATTCAATTCATGAC
ATCGTGCTTTCCTACAACACTAGTGATAGCACAGTATTTCCTGGTGCTGTA
GCTAAAGGAGTTCATACTGTTAAAAATCCTGAGAATTTCAAAGTTCCATTG
GATGTCATCTTTAAGTGCAATAGTGTTTTAACTTACAACCTGACTCCTGTC
GTTCAGAAATATTGGGGTATTCACCTGCAAGCTTTTGTCCAAAATGGTACA
GTGAGTAAAAATGAACAAGTGTGTGAAGAAGACCAAACTCCCACCACTGT
GGCACCCATCATTCACACCACTGCCCCGTCGACTACAACTACACTCACTCC
AACTTCAACACCCACTCCAACTCCAACTCCAACTCCAACCGTTGGAAACT
ACAGCATTAGAAATGGCAATACTACCTGTCTGCTGGCTACCATGGGGCTG
CAGCTGAACATCACTGAGGAGAAGGTGCCTTTCATTTTTAACATCAACCCT
GCCACAACCAACTTCACCGGCAGCTGTCAACCTCAAAGTGCTCAACTTAG
GCTGAACAACAGCCAAATTAAGTATCTTGACTTTATCTTTGCTGTGAAAAA
TGAAAAACGGTTCTATCTGAAGGAAGTGAATGTCTACATGTATTTGGCTA
ATGGCTCAGCTTTCAACATTTCCAACAAGAACCTTAGCTTCTGGGATGCCC
CTCTGGGAAGTTCTTATATGTGCAACAAAGAGCAGGTGCTTTCTGTGTCTA
GAGCGTTTCAGATCAACACCTTTAACCTAAAGGTGCAACCTTTTAATGTGA
CAAAAGGACAGTATTCTACAGCTGAGGAATGTGCTGCTGACTCTGACCTC
AACTTTCTTATTCCTGTTGCAGTGGGTGTGGCCTTGGGCTTCCTTATAATTG
CTGTGTTTATATCTTACATGATTGGAAGACGGAAAAGTCGTACTGGTTATC
AGTCTGTC.
CD206 target
(SEQ ID NO: 2)
TGCTCTCCGGGGGCGAAAGTCAGGTGC
mIgG2b
(SEQ ID NO: 6)
GGGCCCATTTCAACAATCAACCCCTGTCCTCCATGCAAGGAGTGTCACAA
ATGCCCAGCTCCTAACCTCGAGGGTGGACCATCCGTCTTCATCTTCCCTCC
AAATATCAAGGATGTACTCATGATCTCCCTGACACCCAAGGTCACGTGTG
TGGTGGTGGATGTGAGCGAGGATGACCCAGACGTCCAGATCAGCTGGTTT
GTGAACAACGTGGAAGTACACACAGCTCAGACACAAACCCATAGAGAGG
ATTACAACAGTACTATCCGGGTGGTCAGCACCCTCCCCATCCAGCACCAG
GACTGGATGAGTGGCAAGGAGTTCAAATGCAAGGTCAACAACAAAGACC
TCCCATCACCCATCGAGAGAACCATCTCAAAAATTAAAGGGCTAGTCAGA
GCTCCACAAGTATACATCTTGCCGCCACCAGCAGAGCAGTTGTCCAGGAA
AGATGTCAGTCTCACTTGCCTGGTCGTGGGCTTCAACCCTGGAGACATCAG
TGTGGAGTGGACCAGCAATGGGCATACAGAGGAGAACTACAAGGACACC
GCACCAGTCCTGGACTCTGACGGTTCTTACTTCATATACAGCAAGCTCGAT
ATAAAAACAAGCAAGTGGGAGAAAACAGATTCCTTCTCATGCAACGTGAG
ACACGAGGGTCTGAAAAATTACTACCTGAAGAAGACCATCTCCCGGTCTC
CGGGT.

### Example 1: Determination of specificity of Precision Peptide In Vivo and Generation of CD206-Positive M2 Macrophage-Specific Exosomes

### Methods and Materials

To assess in vivo targeting potential, rhodamine-labeled precision peptide (red) was injected intravenously (IV) in metastatic syngeneic murine breast cancer (4T1) bearing Balb/C mice. Three hours after injection, all animals were euthanized, and lungs, spleen and tumors were collected for immune-histochemical analysis. Frozen sections from the collected tissues were stained for CD206 (fluorescein, FITC) and counter stained with DAPI. To confer targeting potentiality, precision peptide for CD206-positive TAMs was fused to the extra-exosomal N-terminus of murine Lamp2b.

### Results

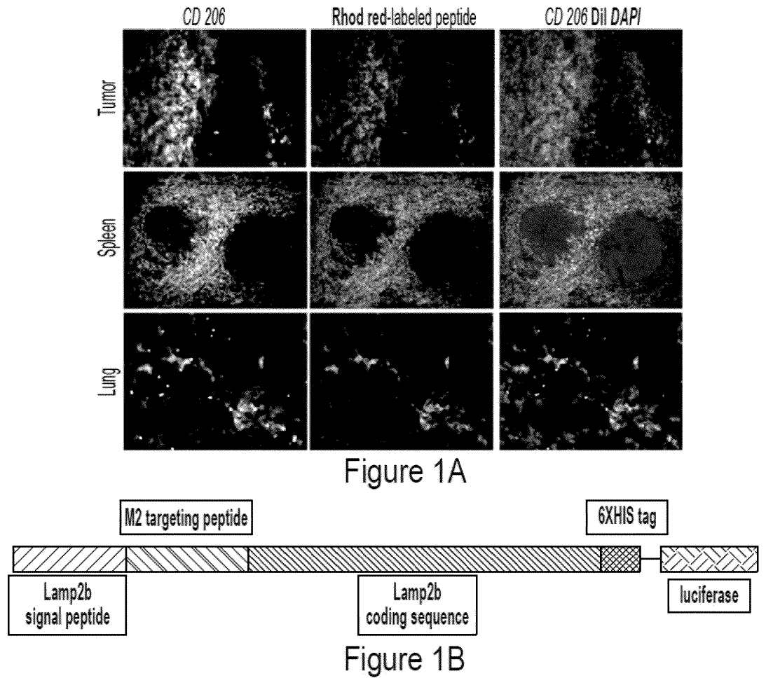

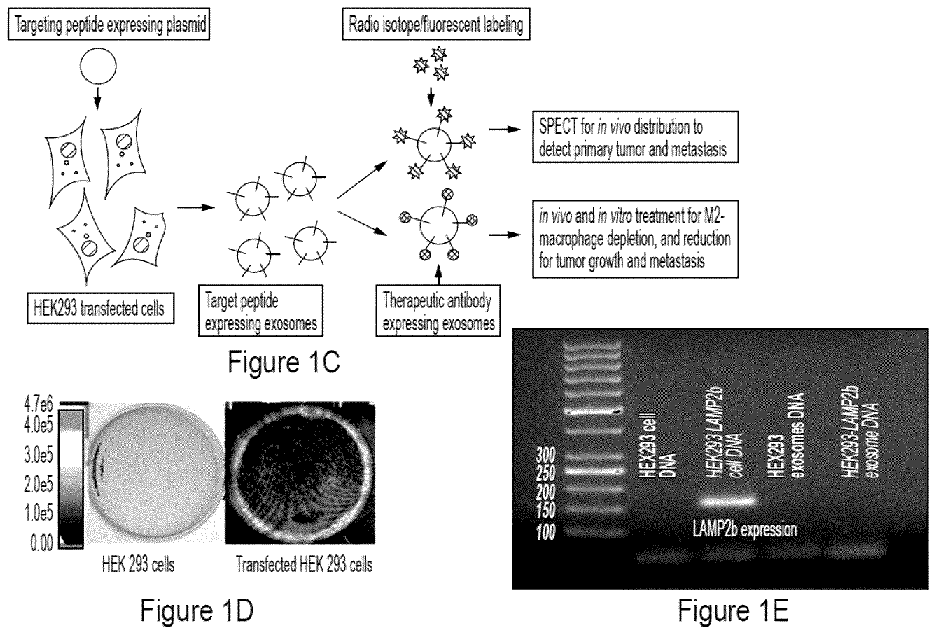

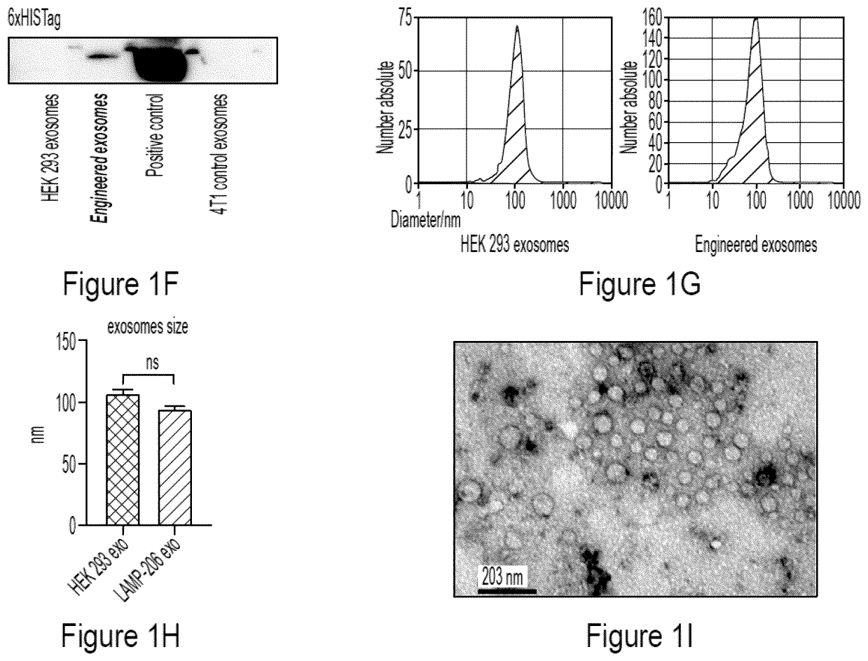

FIG. 1 represents generation of engineered exosomes expressing CD206-positive M2 macrophage-specific peptide along with Lamp2b. FIG. 1a exhibits immunofluorescence staining of tumor, spleen and lungs sections from 4T1 tumor-bearing mice showing co-localization of Rhodamine red-labeled targeting peptide (injected i.v.) and FITC labeled CD206-positive M2-macrophages. Nuclei were visualized by DAPI staining (blue). FIG. 1b is a schematic representation of the modified Lamp2b protein containing CD206 positive M2 macrophage-targeting peptide sequence following signal peptide, and a 6×HIS tag (SEQ ID NO:7) at the C terminus. Luciferase was used as a reporter gene. FIG. 1c is a schematic diagram showing generation of CD206+ M2-macrophage targeting engineered exosomes for diagnostic and therapeutic purpose. FIG. 1d represents in vitro study showing luciferase activity of transfected HEK293 cells. FIG. 1e is agarose gel electrophoresis showing confirmation of targeting peptide sequence insert in transfected HEK293 cells. FIG. 1f is a Western blot image showing anti-His tag antibody positivity in engineered exosomal protein content. FIG. 1g and FIG. 1h showing size distribution by nanoparticle tracking assay (NTA) of the HEK293 exosomes and engineered exosomes, respectively. Quantitative data are expressed in mean±SEM. FIG. 1i illustrates a transmission electron microscopy image for engineered exosomes, (Scale bar depicts 200 nm) showing characteristic round morphology and size without any deformity.

### Example 2: Targeting Potential of CD206-Positive M2-Macrophage-Specific exosomes

### Methods and Materials

To assess targeting ability of the engineered exosomes, mouse RAW264.7 macrophages towards M2-macrophages was differentiated by treating them with IL-4 and IL-3 in vitro. The cells were co-cultured with DiI-labeled (red) engineered exosomes for 4 hours followed by immunofluorescence staining for CD206-positive cells (FITC) and DAPI for nuclei.

### Results

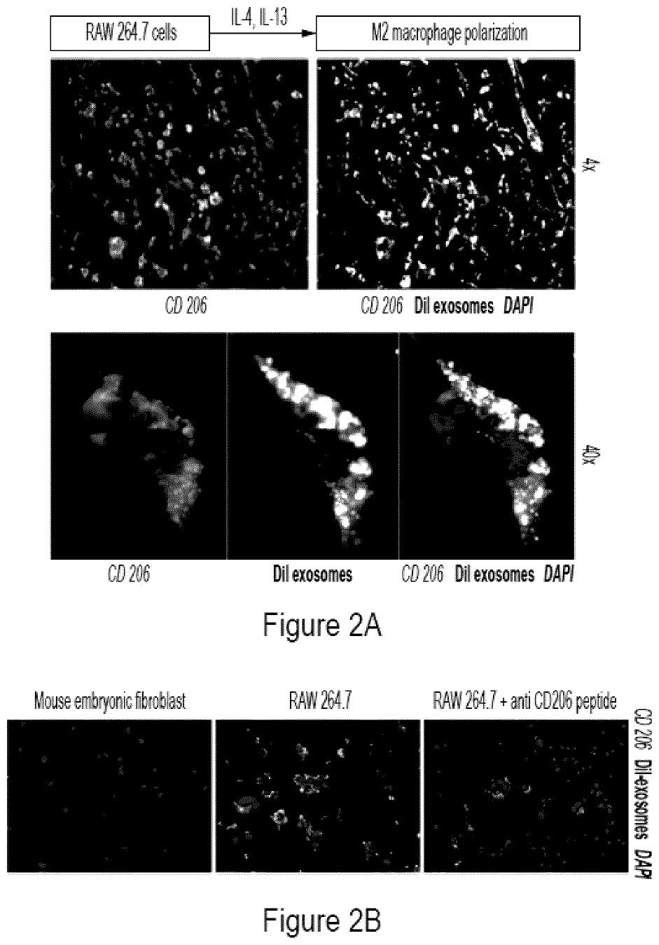

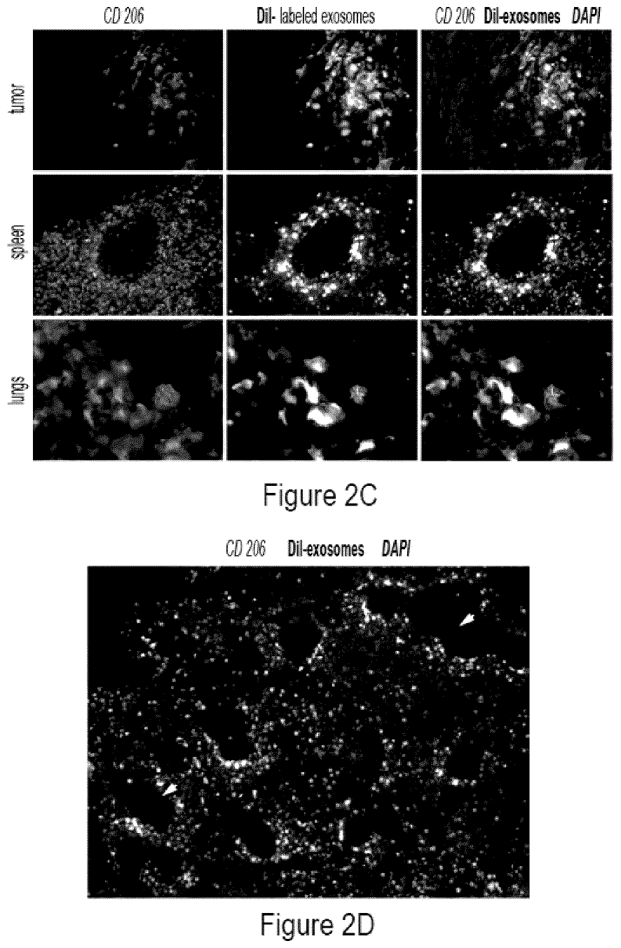

FIG. 2 represents targeting efficiency and specificity of CD206-positive M2 macrophage-specific exosomes. FIG. 2a exhibits immunofluorescence staining showing targeting potential of DiI-labeled (red) engineered exosomes. RAW264.7 mouse macrophages were differentiated to CD206-positive (FITC) cells by treating with interleukin-4 and interleukin-13. Nuclei were visualized by DAPI staining (blue). FIG. 2b exhibits immunofluorescence staining of mouse embryonic fibroblasts (MEFs) and RAW264.7 cells treated with or without anti-CD206 peptide, co-cultured with DiI-labeled (red) engineered exosomes. MEFs were negative for CD206 (FITC) staining and did not take up the exosomes. Engineered exosomes bound to the CD206+RAW264.7 cells, that was prevented by anti-CD206 peptide treatment. FIG. 2c exhibits immunofluorescence staining of tumor, spleen and lungs sections from 4T1 tumor-bearing mice showing co-localization of rhodamine red-labeled targeting exosomes (injected i.v.) and FITC labeled CD206-positive M2-macrophages. Nuclei were visualized by DAPI staining (blue). FIG. 2d exhibits stitched images for extended view of splenic section showing engineered exosomes were not taken up by T-lymphocytes and B-lymphocytes in splenic white pulp (white arrows).

### Example 3: Detection and Quantification of In Vivo Distribution of CD206-Positive M2 Macrophages Targeting Exosomes

### Methods and Materials

To investigate the validity of engineered exosomes as an imaging probe to determine the distribution of M2-macrophages, FDA approved clinically relevant SPECT scanning and labeling with ¹¹¹In-oxine was used according to our previous study (Arbab et al., BMC Med. Imaging 2012, 12, 33). ¹¹¹In-oxine-labeled non-engineered control exosomes (HEK293 exo) in metastatic (4T1) mouse breast cancer models, and engineered exosomes (M2-targeting exo) expressing precision peptide treated with either vehicle or clodronate liposome (Clophosome®-A) 24 hours before the IV administration of ¹¹¹In-oxine-labeled exosomes and SPECT studies was used. Clophosome®-A is composed of anionic lipids and depletes more than 90% macrophages in spleen after a single intravenous injection (Li et al., Scient. Rep. 2016, 6, 22143-22143; Kobayashi et al., J. Biol. Chem. 2015, 290, 12603-12613). Clophosome®-A is not approved for human studies, and it is for experimental use only. Similar to the previously-mentioned, ¹³¹I-labeled exosomes (Rashid et al., Nanomed: Nanotechnol., Biol. Med. 2019, 21, 102072), prior to IV injection into mice for biodistribution, the labeling efficiency of ¹¹¹In-oxine to the engineered exosomes and serum stability of binding by thin layer paper chromatography (TLPC) was checked.

### Results

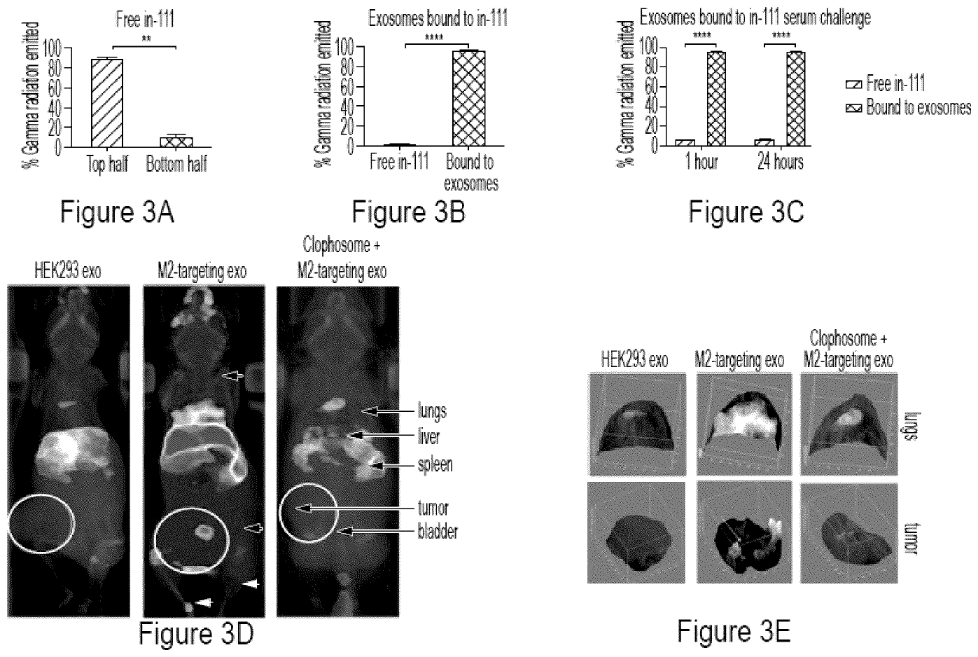

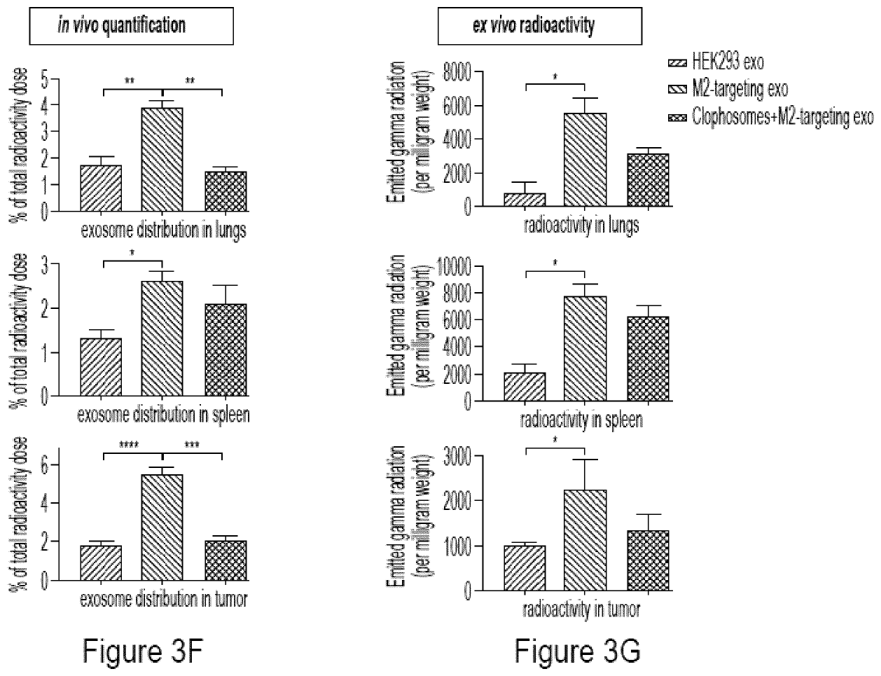

FIG. 3 represents detection and quantification of biodistribution of ¹¹¹In-oxine-labeled exosomes targeting CD206-positive M2 macrophages. FIG. 3a shows a major proportion of the free ¹¹¹In-oxine measured in the bottom to the top half of the thin layer paper chromatography (TLPC) paper, confirming the efficacy of the eluent. FIG. 3b shows binding of ¹¹¹In-oxine to engineered exosomes was validated as shown by a lower percentage of ¹¹¹In-oxine (free, dissociated) measured in the top of the paper, compared to the amount remaining in the bottom, which represented the ¹¹¹In-oxine-labeled exosomes. FIG. 3c shows serum stability of ¹¹¹In-oxine bound engineered exosomes was higher compared with the small amount of free ¹¹¹TH-oxine disengaged from the bound exosomes. FIG. 3d illustrates in vivo SPECT/CT images (coronal view) after 3 hrs of intravenous injection showed significant accumulation of M2-targeting exo in tumor, lung, spleen, lymph node and bones. ¹¹¹In-oxine-labeled non-targeting exosomes (HEK293 exo) and CD206-positive M2-macrophage targeting exosomes (M2-targeting exo) were injected into the 4T1 tumor-bearing mice. One group was treated with Clophosome® to deplete macrophages. Yellow and green arrows denote lymph node and bone metastasis, respectively. FIG. 3e illustrates 3D surface images showing M2-targeting exo are profoundly distributed in both lung and tumor area compared to the group injected with HEK293 exo and pre-treated with Clophosome®. Yellow arrow indicates the tumor center. FIG. 3f shows quantification of in vivo radioactivity in lungs, spleen and tumor. FIG. 3g shows ex vivo radioactivity quantification in lungs, spleen and tumor. Quantitative data are expressed in mean±SEM. *P<0.05, **P<0.01, ***P<0.001, ****P<0.0001. n=3.

### Example 4: Generation of CD206-Positive M2 Macrophage-Targeting Therapeutic Exosomes

### Methods and Materials

Following the confirmation of targeting potential of engineered exosomes for diagnostic purpose, the exosomes as therapeutic carriers was further utilized. Fc portion of mouse IgG2b next to the targeting precision peptide with a small linker with the purpose of inducing ADCC was conjugated. 6×His tag (SEQ ID NO:7) and luciferase were incorporated as reporter genes similar to the previous construct.

### Results

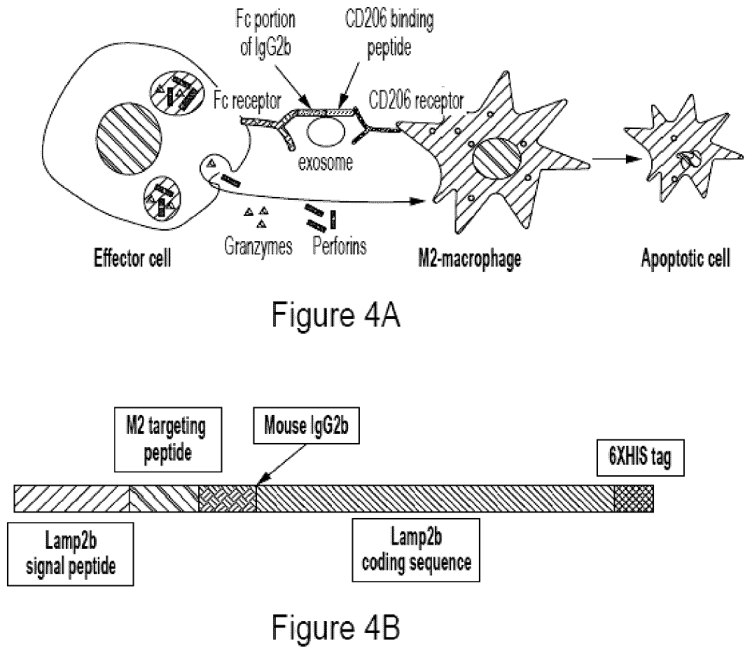

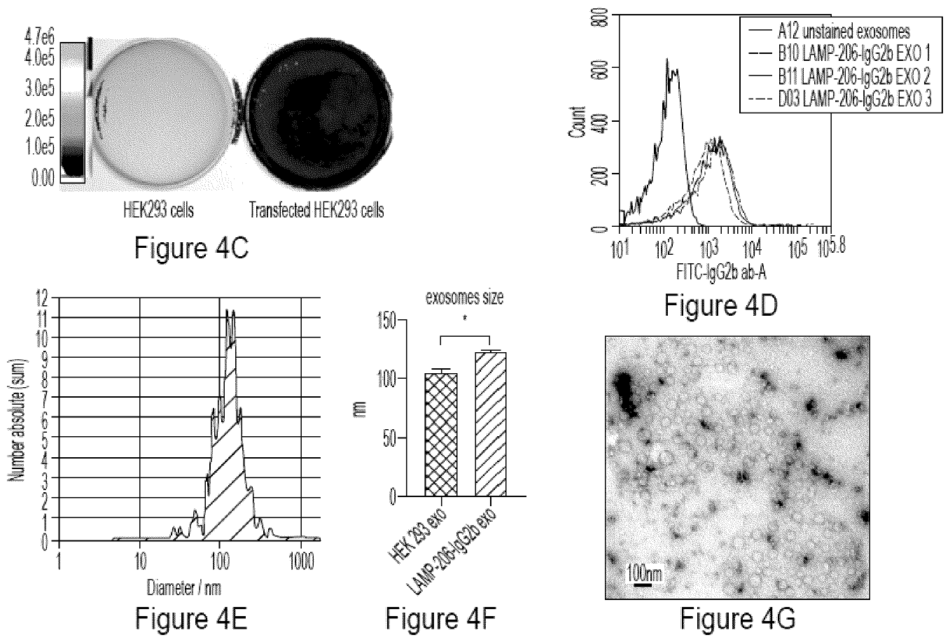

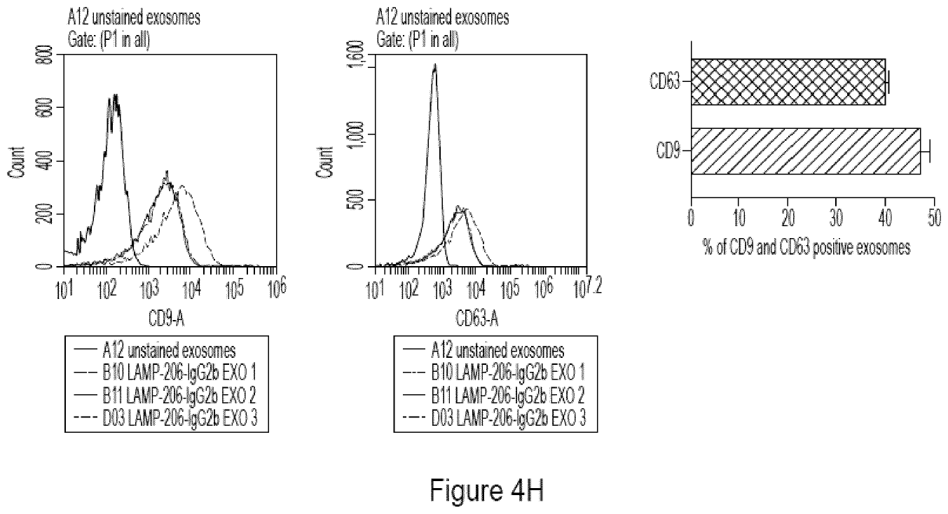

FIG. 4 represents generation of CD206-positive M2 macrophage-targeting therapeutic exosomes to induce antibody-dependent cell-mediated cytotoxicity. FIG. 4a illustrates schematic diagram showing the proposed mechanism of engineered exosome-based antibody-dependent cellular cytotoxicity. FIG. 4b illustrates schematic representation of the plasmid construct containing modified Lamp2b protein with CD206-targeting sequence conjugated with Fc segment of mouse IgG2b. FIG. 4c demonstrates confirmation of luciferase activity by transfected HEK293 cells. FIG. 4d shows flow cytometry analysis for validating the expression of Fc segment of mouse IgG2b on the surface of engineered exosomes. Three different engineered exosome samples were used for the flow cytometry. FIG. 4e shows concentration and size distribution of the engineered therapeutic exosomes by nanoparticle tracking assay (NTA). FIG. 4f shows mean diameter of engineered exosomes was significantly larger than non-engineered exosomes. FIG. 4g illustrates transmission electron microscopy image for engineered therapeutic exosomes, (Scale bar depicts 100 nm) showing distinctive round morphology and size without any distortion. FIG. 4h shows flow-cytometry analysis of exosomal markers CD9 and CD63 for the engineered therapeutic exosomes. Three different engineered exosome samples were used for the flow cytometry.

### Example 5: Induction of Cytotoxicity and Depletion of M2-Macrophages by Engineered Therapeutic Exosomes

### Methods and Materials

To ascertain the capacity of therapeutic exosomes for instigating ADCC, the CFSE-labeled (green) RAW264.7 macrophages was treated with non-therapeutic CD206-positive cell-targeting exosomes (LAMP-206 exo) or CD206-positive cell-targeting therapeutic exosomes (LAMP-206-IgG2b exo), and without any exosome treatment (control) for 48 hours in presence of normal mouse splenic mononuclear cells.

### Results

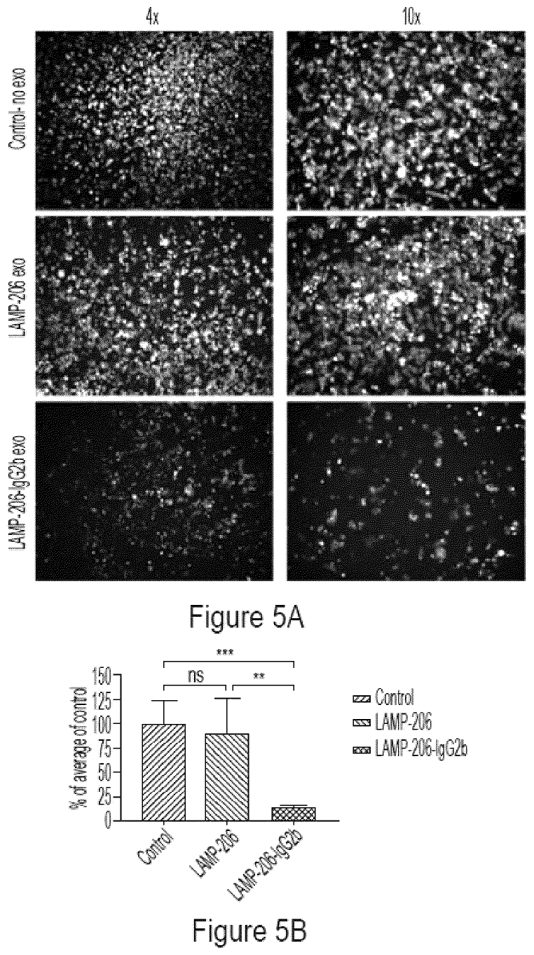

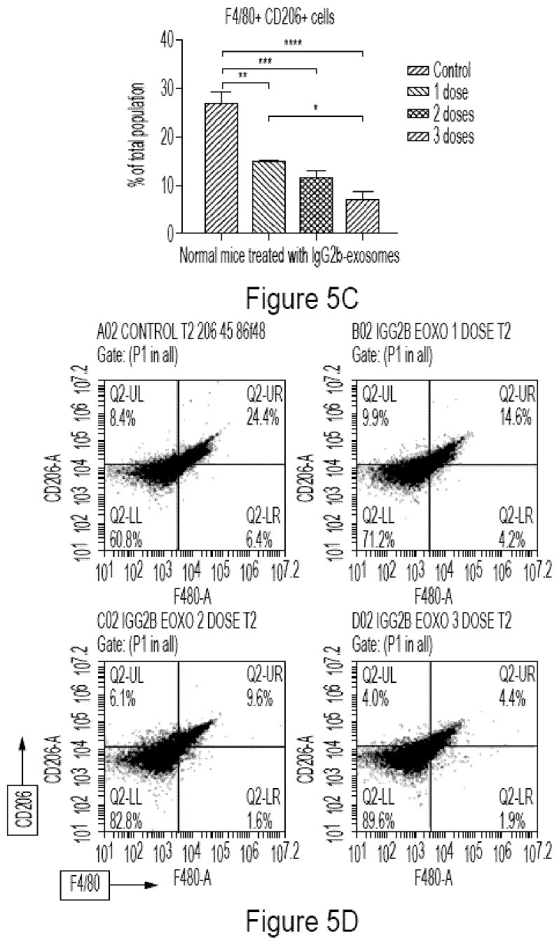

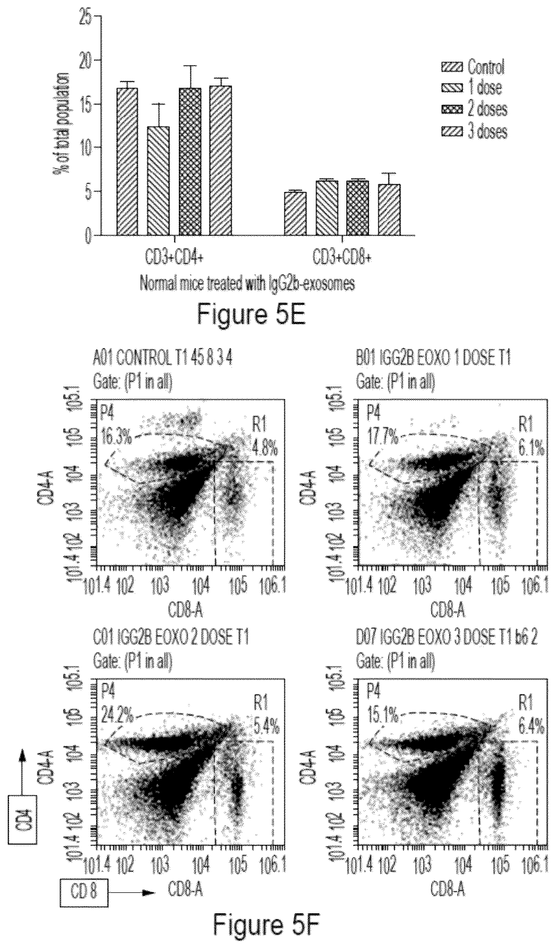

FIG. 5 represents therapeutic efficiency and specificity of engineered therapeutic exosomes in depleting M2-macrophages both in vitro and in vivo. FIG. 5a illustrates CFSE-labeled (green) RAW264.7 mouse macrophages were co-cultured with non-therapeutic CD206-positive cell-targeting exosomes (LAMP-206 exo) or CD206-positive cell-targeting therapeutic exosomes (LAMP-206-IgG2b exo), and without treatment (control) for 48 hours in presence of splenic immune cells from normal mice. Fluorescence microscopic images showed decrease in cell number and increased floating dead cells in LAMP-206-IgG2b exo group compared to other groups. FIG. 5b shows measured fluorescence intensity of the above-mentioned conditions showed significant decrease in LAMP-206-IgG2b exo group compared to other groups. FIG. 5c and FIG. 5d exhibits normal Balb/c mice were treated with one, two or three doses of engineered therapeutic exosomes expressing Fc portion of mouse IgG2b. Flow-cytometry analysis of splenic cells showing dose-dependent decline of F4/80 and CD206-positive M2-macrophage population. FIG. 5e and FIG. 5f illustrates flow-cytometry analysis of splenic cells showing no significant change in both CD4 and CD8-positive T-cell population after treating the mice with different doses of therapeutic exosomes. Quantitative data are expressed in mean±SEM. *P<0.05, **P<0.01, ***P<0.001, ****P<0.0001. n=5.

### Example 6: Treatment With Engineered Therapeutic Exosomes Prevent Tumor Growth and Early Metastasis Increasing Survival

### Methods and Materials

Furthermore, in vivo distribution of the precision peptide after therapeutic exosome treatment in mouse tumor model to see if the treatment can attenuate distribution of the peptide in M2-macrophage prevalent areas was determined. Tumor cells were implanted subcutaneously on the flanks of mice. After 3 weeks of tumor growth, one group of mice was treated with engineered therapeutic exosomes for one week (3 doses), and another group of mice was without treatment. 6-Hydrazinopyridine-3-carboxylic acid (HYNIC) was conjugated with the precision peptide and labeled with technetium-99m (99mTc). 99mTc-labeled peptide was injected into both groups of mice and after 3 hours CT followed by SPECT images were acquired.

### Results

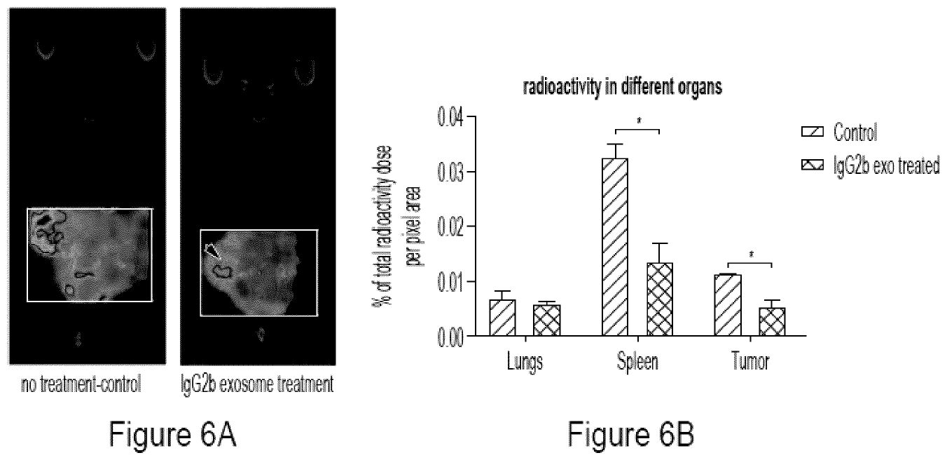

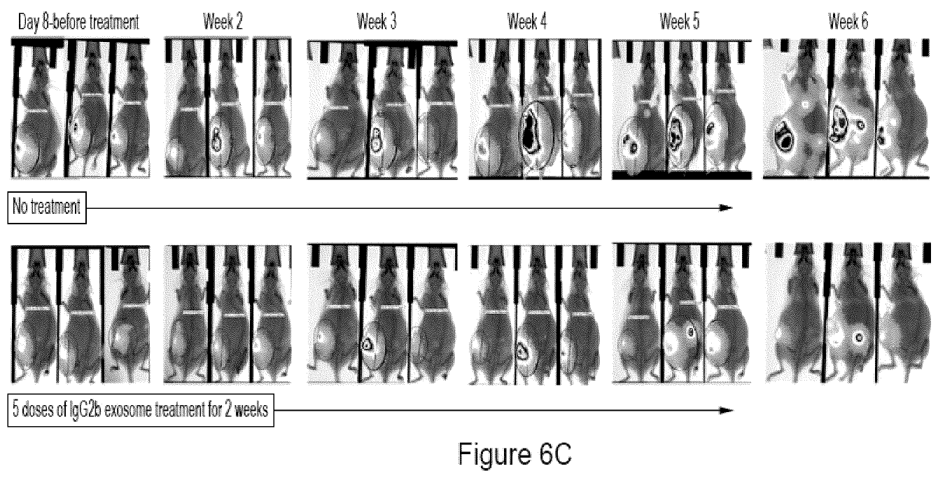

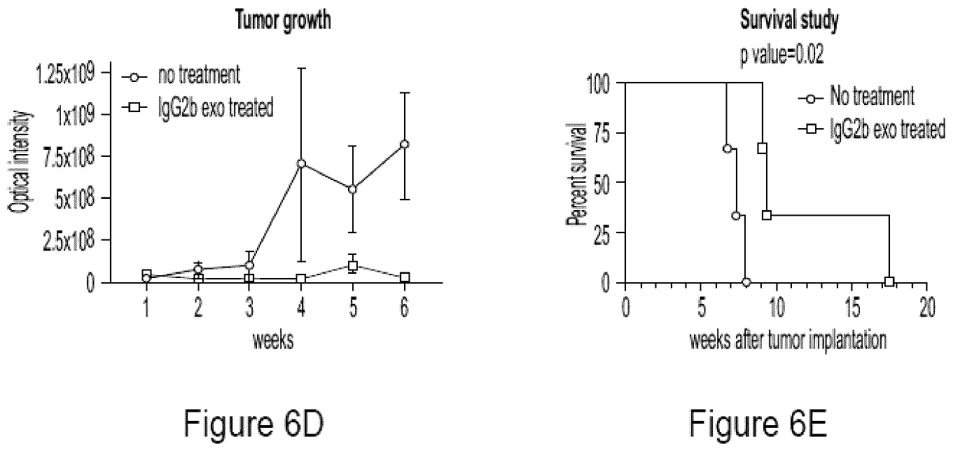

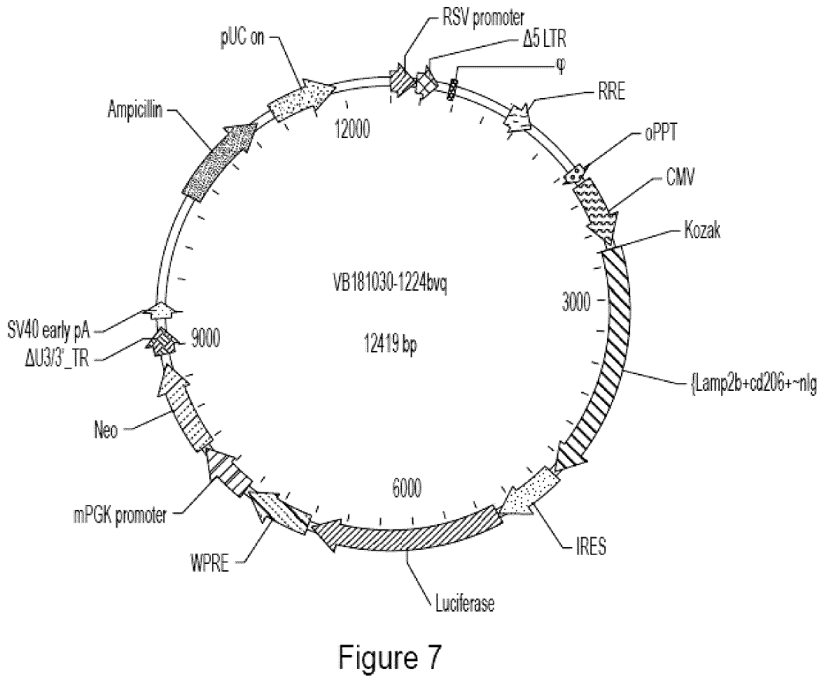

FIG. 6 represents treatment of 4T1 tumor-bearing animals with therapeutic engineered exosomes prevent tumor growth and metastasis, and improve survival by depleting M2-macrophages. FIG. 6a and FIG. 6b illustrates reconstructed and co-registered in vivo SPECT/CT images (coronal view) and quantification of subcutaneous syngeneic tumor-bearing animals (on the flank) injected with the 99mTc-labeled precision peptide after three hours. Group treated with therapeutic exosomes showed lesser level of radioactivity in tumor (yellow arrow) and spleen compared to untreated control group. Quantitative data are expressed in mean±SEM, *P<0.05. n=3. FIG. 6c displays optical images of 4T1 tumor-bearing animals treated with engineered therapeutic exosomes (lower panel) or without treatment (control), showing decreased tumor growth in treated animals compared to control group. Metastatic foci in control group was detected (yellow arrows) as early as fourth week, whereas no metastasis was detected in treated animals after 6 weeks. FIG. 6d illustrates quantification of optical density of the tumor area also showed decreased tumor growth in treated group compared to control group. Quantitative data are expressed in mean±SEM. n=3. FIG. 6e shows Kaplan-Meier plot showing prolonged survival of the mice treated with therapeutic engineered exosomes.

### Example 7: Neuron Targeting Peptide (Rabies Virus Glycoprotein) Based Engineered Exosomes to Deliver Ngb (Neuroglobin) Into the Neurons

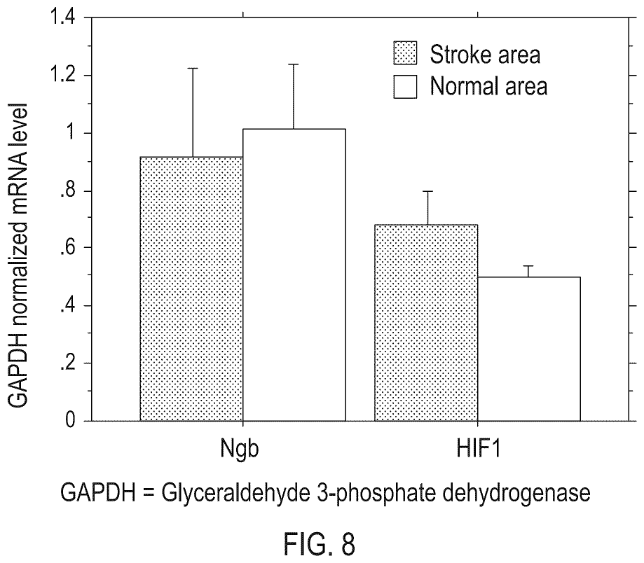

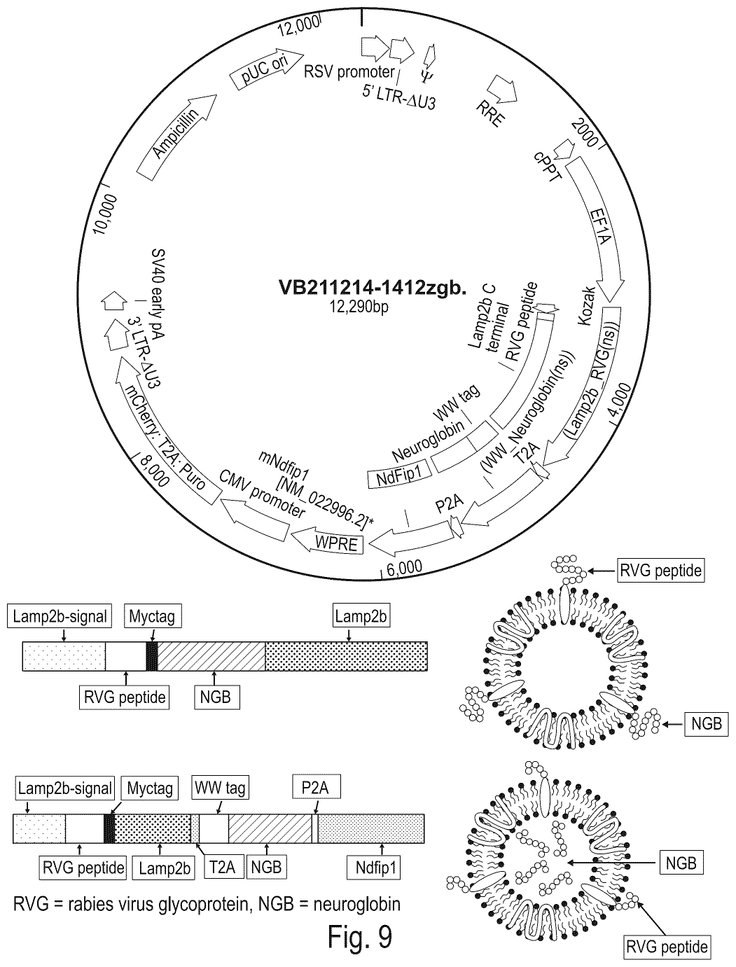

In vitro and in vivo studies showed that Ngb protects the brain from hypoxic/ischemic and oxidative stress-related insults due to its capacity to bind oxygen reversibly (FIG. 8). Therefore, the Fc gamma-receptor (FcR) based targeting exosome platform was used to develop neuron targeting engineered exosomes to deliver Neuroglobin (Ngb) (FIG. 9).

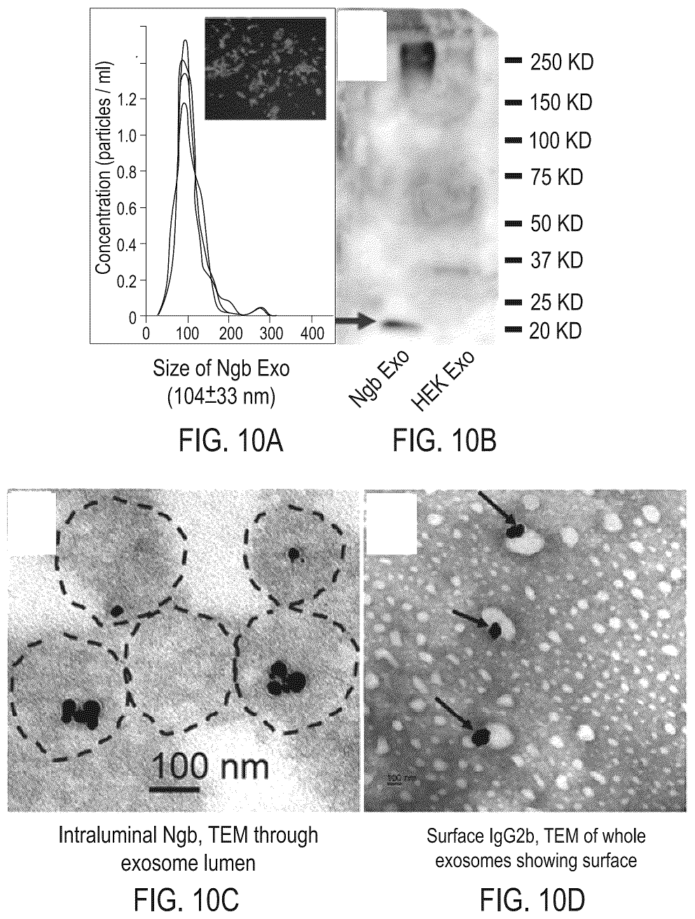

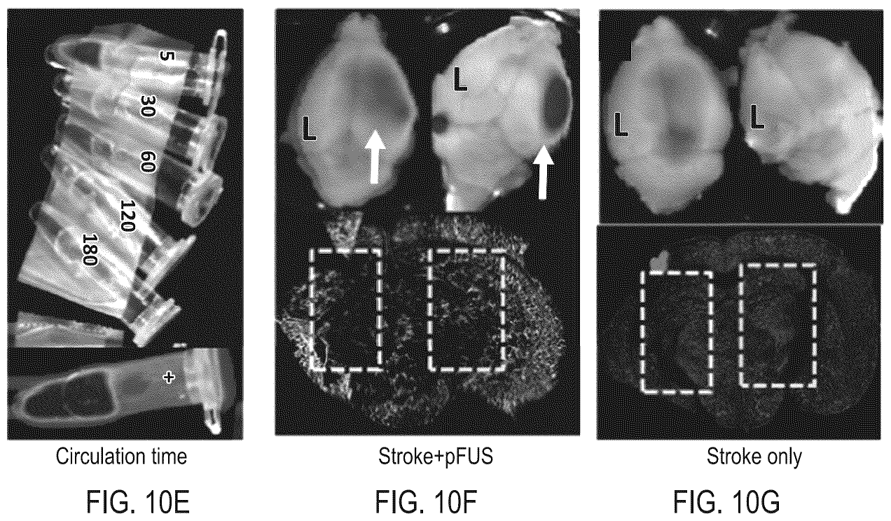

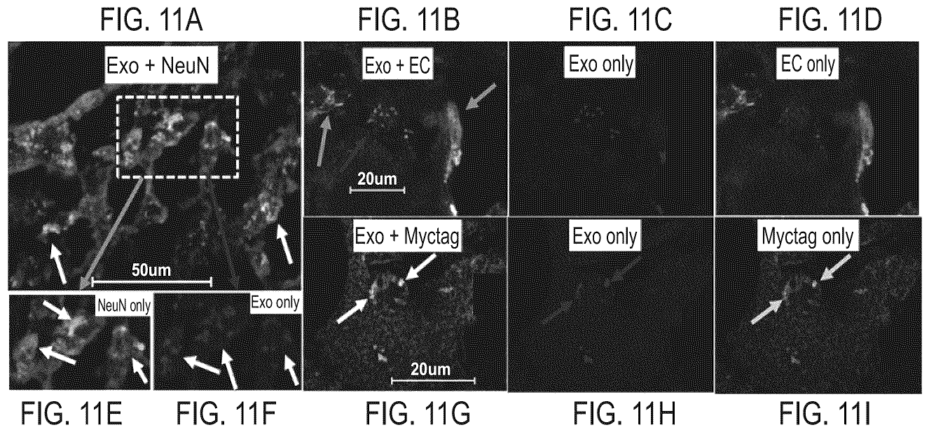

Using the 10 to 12 months old C57BL/6 mouse stroke model, neuron targeting-Ngb exosomes were IV administered 24hrs after stroke soon after focal ultrasound and brains were collected after 3 hrs of treatment (FIGS. 10A-10G). Specific targeting of neurons was shown in the stroke area (FIGS. 11A-11I).

### Materials and Methods:

Engineered exosomes carrying RVG plus Ngb were developed using our platforms. Following the development of stroke and initial MRI, randomly selected animals received pFUS followed by IV administration of fluorescent dye labeled engineered exosomes carrying RVG plus Ngb 24 hrs after the development of stroke. 2 to 3 hrs. after IV injection of engineered exomes, animals were euthanized and perfused with ice cold PBS. Then the brains were collected, fixed and frozen sections were made for immunohistochemistry to determine the site of accumulation of IV administered exosomes in the stroke areas and to show than RVG expressing exosomes binds specifically to neurons and the exosomes are not binding non-specifically to endothelial cells.

For surface expressing RVG plus NGB Sequences: “Lamp2b signaling-linker-RGV-linker-NGB-linker-Myc-linker-Lamp2b c-terminus-Stop codon:

(SEQ ID NO: 8)
ATGTGCCTCTCTCCGGTTAAAGGCGCAAAGCTCATCCTGATCTTTCTGT
TCCTAGGAGCCGTTCAGTCCAATGCAAGCGCTTACACCATCTGGATGCC
CGAGAACCCCAGGCCCGGCACCCCCTGCGACATCTTCACCAACAGCAGG
GGCAAGAGGGCCAGCAACGGCAGCGCTATGGAGCGCCCGGAGTCAGAGC
TGATCCGGCAGAGCTGGCGGGTAGTGAGCCGCAGCCCTCTGGAACATGG
CACTGTCCTGTTCGCCAGGCTCTTCGCCCTGGAACCCAGCCTGCTGCCT
CTCTTCCAGTACAATGGCCGCCAGTTCTCCAGCCCTGAGGACTGTCTCT
CCTCTCCAGAATTCCTGGACCACATTAGGAAGGTGATGCTAGTGATTGA
TGCTGCAGTGACCAACGTGGAGGACCTGTCTTCATTGGAGGAGTACCTG
ACCAGCTTGGGCAGGAAGCATCGGGCAGTGGGAGTGAGGCTCAGCTCCT
TCTCGACAGTAGGCGAGTCCCTGCTCTACATGCTGGAGAAGTGCCTGGG
TCCCGACTTTACACCAGCTACAAGGACCGCCTGGAGCCGACTCTACGGA
GCTGTGGTGCAAGCCATGAGCCGAGGCTGGGATGGGGAGGGATCCGAGC
AGAAACTCATCTCTGAAGAGGATCTGCCCGGGTTGATAGTTAATTTGAC
AGATTCAAAGGGTACTTGCCTTTATGCAGAATGGGAGATGAATTTCACA
ATAACATATGAAACTACAAACCAAACCAATAAAACTATAACCATTGCAG
TACCTGACAAGGCGACACACGATGGAAGCAGTTGTGGGGATGACCGGAA
TAGTGCCAAAATAATGATACAATTTGGATTCGCTGTCTCTTGGGCTGTG
AATTTTACCAAGGAAGCATCTCATTATTCAATTCATGACATCGTGCTTT
CCTACAACACTAGTGATAGCACAGTATTTCCTGGTGCTGTAGCTAAAGG
AGTTCATACTGTTAAAAATCCTGAGAATTTCAAAGTTCCATTGGATGTC
ATCTTTAAGTGCAATAGTGTTTTAACTTACAACCTGACTCCTGTCGTTC
AGAAATATTGGGGTATTCACCTGCAAGCTTTTGTCCAAAATGGTACAGT
GAGTAAAAATGAACAAGTGTGTGAAGAAGACCAAACTCCCACCACTGTG
GCACCCATCATTCACACCACTGCCCCGTCGACTACAACTACACTCACTC
CAACTTCAACACCCACTCCAACTCCAACTCCAACTCCAACCGTTGGAAA
CTACAGCATTAGAAATGGCAATACTACCTGTCTGCTGGCTACCATGGGG
CTGCAGCTGAACATCACTGAGGAGAAGGTGCCTTTCATTTTTAACATCA
ACCCTGCCACAACCAACTTCACCGGCAGCTGTCAACCTCAAAGTGCTCA
ACTTAGGCTGAACAACAGCCAAATTAAGTATCTTGACTTTATCTTTGCT
GTGAAAAATGAAAAACGGTTCTATCTGAAGGAAGTGAATGTCTACATGT
ATTTGGCTAATGGCTCAGCTTTCAACATTTCCAACAAGAACCTTAGCTT
CTGGGATGCCCCTCTGGGAAGTTCTTATATGTGCAACAAAGAGCAGGTG
CTTTCTGTGTCTAGAGCGTTTCAGATCAACACCTTTAACCTAAAGGTGC
AACCTTTTAATGTGACAAAAGGACAGTATTCTACAGCTGAGGAATGTGC
TGCTGACTCTGACCTCAACTTTCTTATTCCTGTTGCAGTGGGTGTGGCC
TTGGGCTTCCTTATAATTGCTGTGTTTATATCTTACATGATTGGAAGAC
GGAAAAGTCGTACTGGTTATCAGTCTGTCTAA

For surface expressing RVG but intraluminal NGB Sequences: “Lamp2b signaling-linker-RGV-linker-Myc-linker-Lamp2b c-terminus-EcoRV-T2A-WW tag-NGB-P2A-Ndfib1-EcoRV-S top codon:

(SEQ ID NO: 9)
atgtgcctctctccggttaaaggcgcaaagctcatcctgatctttctgt
tcctaggagccgttcagtccaatgcaagcgcttacaccatctggatgcc
cgagaaccccaggcccggcaccccctgcgacatcttcaccaacagcagg
ggcaagagggccagcaacggcggatccgagcagaaactcatctctgaag
aggatctgcccgggttgatagttaatttgacagattcaaagggtacttg
cctttatgcagaatgggagatgaatttcacaataacatatgaaactaca
aaccaaaccaataaaactataaccattgcagtacctgacaaggcgacac
acgatggaagcagttgtggggatgaccggaatagtgccaaaataatgat
acaatttggattcgctgtctcttgggctgtgaattttaccaaggaagca
tctcattattcaattcatgacatcgtgctttcctacaacactagtgata
gcacagtatttcctggtgctgtagctaaaggagttcatactgttaaaaa
tcctgagaatttcaaagttccattggatgtcatctttaagtgcaatagt
gttttaacttacaacctgactcctgtcgttcagaaatattggggtattc
acctgcaagcttttgtccaaaatggtacagtgagtaaaaatgaacaagt
gtgtgaagaagaccaaactcccaccactgtggcacccatcattcacacc
actgccccgtcgactacaactacactcactccaacttcaacacccactc
caactccaactccaactccaaccgttggaaactacagcattagaaatgg
caatactacctgtctgctggctaccatggggctgcagctgaacatcact
gaggagaaggtgcctttcatttttaacatcaaccctgccacaaccaact
tcaccggcagctgtcaacctcaaagtgctcaacttaggctgaacaacag
ccaaattaagtatcttgactttatctttgctgtgaaaaatgaaaaacgg
ttctatctgaaggaagtgaatgtctacatgtatttggctaatggctcag
ctttcaacatttccaacaagaaccttagcttctgggatgcccctctggg
aagttcttatatgtgcaacaaagagcaggtgctttctgtgtctagagcg
tttcagatcaacacctttaacctaaaggtgcaaccttttaatgtgacaa
aaggacagtattctacagctgaggaatgtgctgctgactctgacctcaa
ctttcttattcctgttgcagtgggtgtggccttgggcttccttataatt
gctgtgtttatatcttacatgattggaagacggaaaagtcgtactggtt
atcagtctgtcgatatcggaagcggagagggcaggggaagtcttctaac
atgcggggacgtggaggaaaatcccggccccatgagcggcctgcccccc
ggctgggaggagaagcaggacgacaggggcaggagctactacgtggacc
acaacagcaagaccaccacctggagcaagcccaccatgcaggacgaccc
caggagcaagatccccgcccacctgaggggcaagaccgacagcaacgac
ctgggccccctgccccccggctgggaggagaggacccacaccgacggca
gggtgttcttcatcaaccacaacatcaagaagacccagtgggaggaccc
caggctgatggagcgcccggagtcagagctgatccggcagagctggcgg
gtagtgagccgcagccctctggaacatggcactgtcctgttcgccaggc
tcttcgccctggaacccagcctgctgcctctcttccagtacaatggccg
ccagttctccagccctgaggactgtctctcctctccagaattcctggac
cacattaggaaggtgatgctagtgattgatgctgcagtgaccaacgtgg
aggacctgtcttcattggaggagtacctgaccagcttgggcaggaagca
tcgggcagtgggagtgaggctcagctccttctcggtgggtagtgggaca
gtaggcgagtccctgctctacatgctggagaagtgcctgggtcccgact
ttacaccagctacaaggaccgcctggagccgactctacggagctgtggt
gcaagccatgagccgaggctgggatggggagggaagcggagccacgaac
ttctctctgttaaagcaagcaggagatgttgaagaaaaccccgggccta
tggccttggcgttggcggcgctggcggccgtggagccggcctgcggcag
cgggtaccagcagttgcagaatgaagaggagcctggggaacctgagcag
actgcaggtgatgctcctccaccatacagcagcatcacggcagagagtg
cagcatattttgactacaaagatgaatctggatttccaaagcccccatc
gtataatgtggctacaacactgcccagttatgacgaggctgagagaacc
aagactgaagctacgatccctttggttcctggaagagatgaagattttg
tgggccgggatgattttgatgatactgaccagctgaggataggaaacga
tgggatttttatgttaacttttttcatggcattcctcttcaactggatt
gggtttttcttgtctttttgcctgaccacctcagctgcgggaaggtatg
gggccatctcaggatttggtctttctctaattaagtggatccttattgt
caggttttccacctatttccctggatactttgatggccagtactggctc
tggtgggtgttcttggttttaggctttctcctgtttctcagaggattta
tcaattacgcaaaagttcggaagatgccagaaactttctcaaatctccc
caggaccagagttctctttatttatgatatctaa

### Results:

Results showed increased accumulation of IV administered engineered exosomes carrying RVG plus Ngb in the stroke areas. Exosomes were seen in the brain parenchyma away from the blood vessels indicating penetration of exosomes through BBB. Exosomes were seen attached with the cells that express neuron marker NeuN. Presence of exosomes were confirmed by fluorescent tag (Dil) as well as Myctag.

### Example 8: Myeloid Cell Targeting Exosomes Target and Kill Myeloid Cells by ADCC

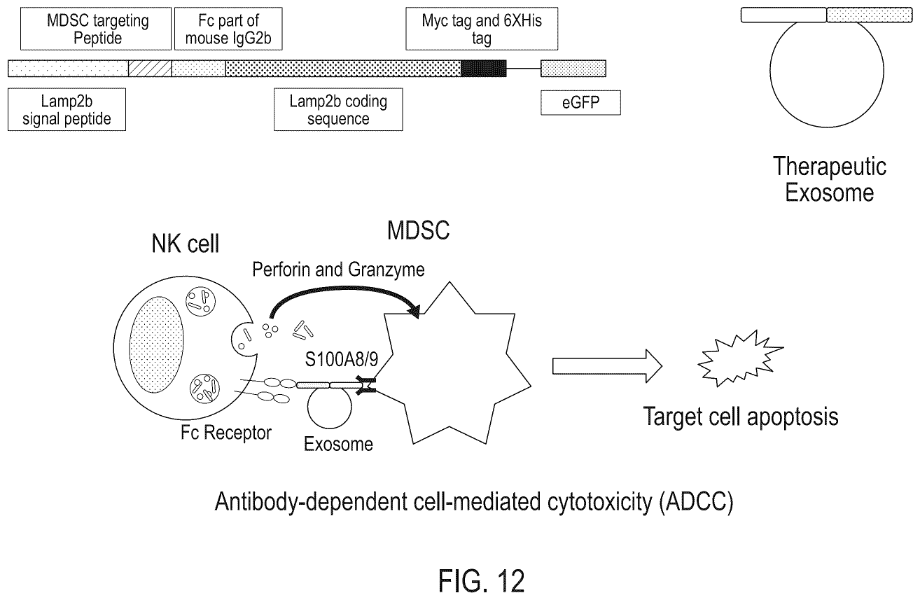

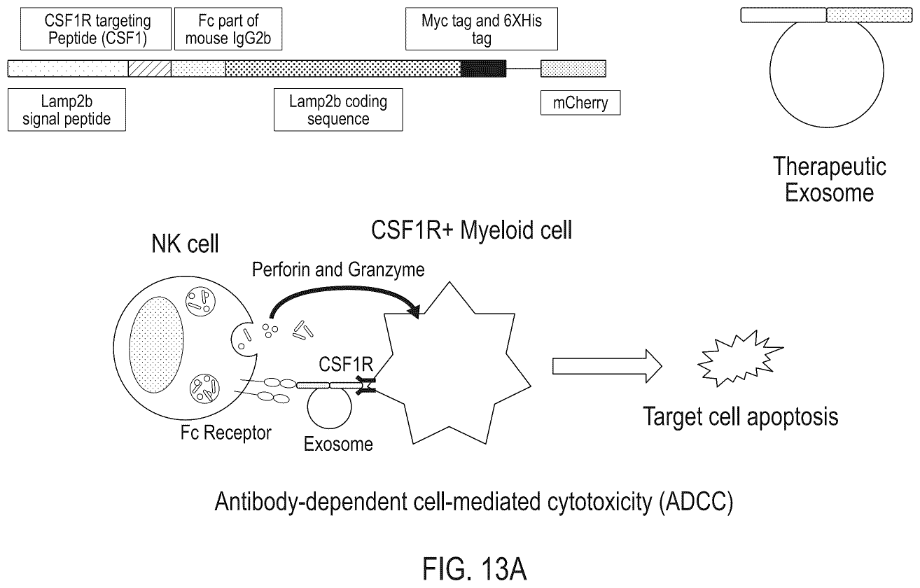

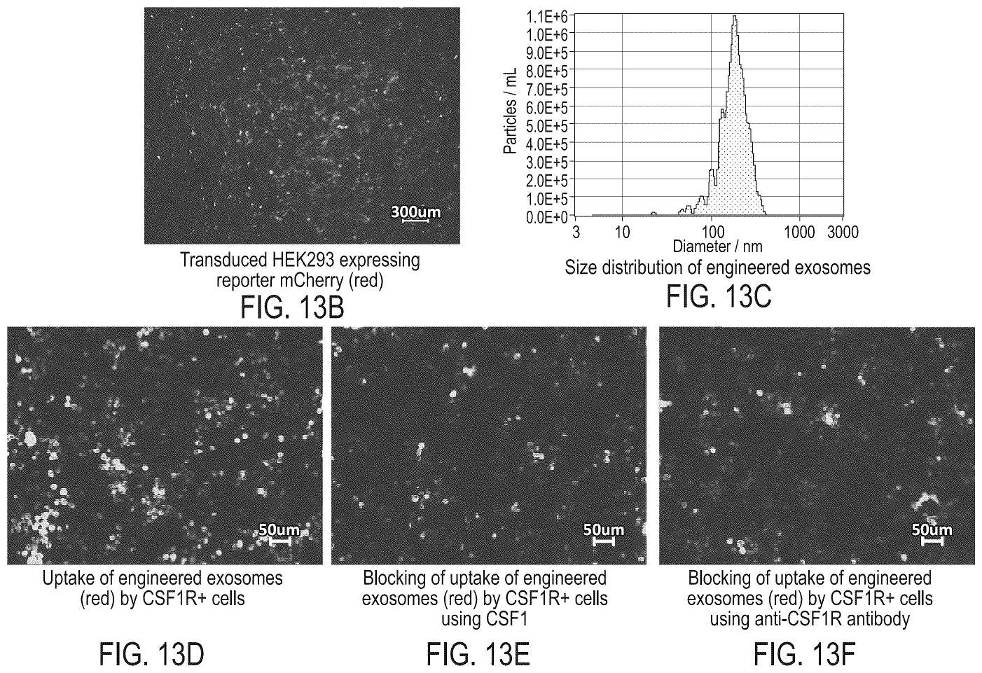

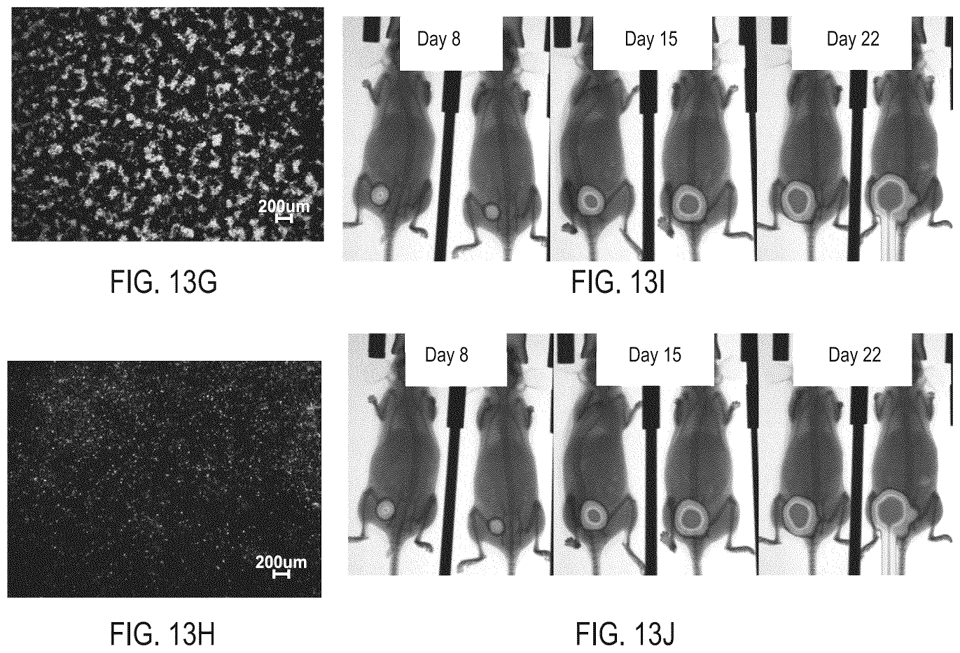

The Fc gamma-receptor (FcR) based targeting exosome platform was used to develop a myeloid-derived suppressor cells (MDSC) targeting peptide engineered exosome (FIG. 12) that targets and kills MDSC by antibody-dependent cell-mediated cytotoxicity (ADCC). Additionally, FIG. 13 shows that the Fc gamma-receptor (FcR) based targeting exosome platform was also used to develop a colony stimulating factor 1 receptor (CSF-1R) targeting peptide that targets and kills myeloid cells by ADCC.

### Materials and Methods:

With our established platform of making engineered exosomes we have used targeting peptides that target either target MDSC or CSF1R+ cells. For therapeutic load to deplete targeted cells we used mouse Fc-portion of IgG2b. With the following nucleic acid sequences, we have created vectors (plasmid). Then we have created Lentiviral vectors (using HEK293TN cells) in the lab using the designed plasmids. Produced Lentiviral vector was used to transduced HEK293 cells and then selected using specific antibiotic. The selected cells are used to make engineered exosomes. As described above, transduced cells are cultured in exosome free complete media and the conditioned media are collected at 24, 48 and 72 hrs for collection of released exosomes. Collected exosomes are subjected to characterization in respect of size, number, and the contents. In vitro studies are performed to determine the killing effect of the engineered exosomes. In vivo studies are done to determine the effect on survival or tumor size following therapies with the engineered exosomes targeting CSF1R+ cells.

Sequences: Lamp2b signaling peptide-linker-mouse CSF1R targeting peptides-linker-mouse IgG2b-linker-Myc-Lamp2b C-terminus-Stop codon:

(SEQ ID NO: 10)
ATGTGCCTCTCTCCGGTTAAAGGCGCAAAGCTCATCCTGATCTTTCTGT
TCCTAGGAGCCGTTCAGTCCAATGCAAGCGCTGGTAACTCGACTATGGG
CAGTGGATCAGAACACTGTAGCCACATGATTGGGAATGGACACCTGAAG
GTCCTGCAGCAGTTGATCGACAGTCAAATGGAGACTTCATGCCAGATTG
CCTTTGAATTTGTAGACCAGGAACAGCTGGATGATCCTGTTTGCTACCT
AAAGAAGGCCTTTTTTCTGGTACAAGACATAATAGATGAGACCATGCGC
TTTAAAGACAACACCCCCAATGCTAACGCCACCGAGAGGCTCCAGGAAC
TCTCCAATAACCTGAACAGCTGCTTCACCAAGGACTATGAGGAGCAGAA
CAAGGCCTGTGTCCGAACTTTCCATGAGACTCCTCTCCAGCTGCTGGAG
AAGATCAAGAACTTCTTTAATGAAACAAAGAATCTCCTTGAAGGCAGTG
GATCTGGTGGCTCGAGTGGATCCGGGCCCATTTCAACAATCAACCCCTG
TCCTCCATGCAAGGAGTGTCACAAATGCCCAGCTCCTAACCTCGAGGGT
GGACCATCCGTCTTCATCTTCCCTCCAAATATCAAGGATGTACTCATGA
TCTCCCTGACACCCAAGGTCACGTGTGTGGTGGTGGATGTGAGCGAGGA
TGACCCAGACGTCCAGATCAGCTGGTTTGTGAACAACGTGGAAGTACAC
ACAGCTCAGACACAAACCCATAGAGAGGATTACAACAGTACTATCCGGG
TGGTCAGCACCCTCCCCATCCAGCACCAGGACTGGATGAGTGGCAAGGA
GTTCAAATGCAAGGTCAACAACAAAGACCTCCCATCACCCATCGAGAGA
ACCATCTCAAAAATTAAAGGGCTAGTCAGAGCTCCACAAGTATACATCT
TGCCGCCACCAGCAGAGCAGTTGTCCAGGAAAGATGTCAGTCTCACTTG
CCTGGTCGTGGGCTTCAACCCTGGAGACATCAGTGTGGAGTGGACCAGC
AATGGGCATACAGAGGAGAACTACAAGGACACCGCACCAGTCCTGGACT
CTGACGGTTCTTACTTCATATACAGCAAGCTCGATATAAAAACAAGCAA
GTGGGAGAAAACAGATTCCTTCTCATGCAACGTGAGACACGAGGGTCTG
AAAAATTACTACCTGAAGAAGACCATCTCCCGGTCTCCGGGTGATATCG
GAGAGCAGAAACTCATCTCTGAAGAGGATCTGTTGATAGTTAATTTGAC
AGATTCAAAGGGTACTTGCCTTTATGCAGAATGGGAGATGAATTTCACA
ATAACATATGAAACTACAAACCAAACCAATAAAACTATAACCATTGCAG
TACCTGACAAGGCGACACACGATGGAAGCAGTTGTGGGGATGACCGGAA
TAGTGCCAAAATAATGATACAATTTGGATTCGCTGTCTCTTGGGCTGTG
AATTTTACCAAGGAAGCATCTCATTATTCAATTCATGACATCGTGCTTT
CCTACAACACTAGTGATAGCACAGTATTTCCTGGTGCTGTAGCTAAAGG
AGTTCATACTGTTAAAAATCCTGAGAATTTCAAAGTTCCATTGGATGTC
ATCTTTAAGTGCAATAGTGTTTTAACTTACAACCTGACTCCTGTCGTTC
AGAAATATTGGGGTATTCACCTGCAAGCTTTTGTCCAAAATGGTACAGT
GAGTAAAAATGAACAAGTGTGTGAAGAAGACCAAACTCCCACCACTGTG
GCACCCATCATTCACACCACTGCCCCGTCGACTACAACTACACTCACTC
CAACTTCAACACCCACTCCAACTCCAACTCCAACTCCAACCGTTGGAAA
CTACAGCATTAGAAATGGCAATACTACCTGTCTGCTGGCTACCATGGGG
CTGCAGCTGAACATCACTGAGGAGAAGGTGCCTTTCATTTTTAACATCA
ACCCTGCCACAACCAACTTCACCGGCAGCTGTCAACCTCAAAGTGCTCA
ACTTAGGCTGAACAACAGCCAAATTAAGTATCTTGACTTTATCTTTGCT
GTGAAAAATGAAAAACGGTTCTATCTGAAGGAAGTGAATGTCTACATGT
ATTTGGCTAATGGCTCAGCTTTCAACATTTCCAACAAGAACCTTAGCTT
CTGGGATGCCCCTCTGGGAAGTTCTTATATGTGCAACAAAGAGCAGGTG
CTTTCTGTGTCTAGAGCGTTTCAGATCAACACCTTTAACCTAAAGGTGC
AACCTTTTAATGTGACAAAAGGACAGTATTCTACAGCTGAGGAATGTGC
TGCTGACTCTGACCTCAACTTTCTTATTCCTGTTGCAGTGGGTGTGGCC
TTGGGCTTCCTTATAATTGCTGTGTTTATATCTTACATGATTGGAAGAC
GGAAAAGTCGTACTGGTTATCAGTCTGTCCATCACCATCACCATCACTA
A

### Results:

FIGS. 13B-13J show successful transduction and selection of transgenic cells (mCherry positive cells). NTA analysis shows average diameter of the engineered exosomes is 178.6 nm. Specific of the engineered exosomes to target CSF1R+ cells were tested by blocking the interaction of CSF1-CSF1R axis using CAF1 or antiCSF1R antibody. Both conditions significantly decreased the uptake of the exosomes compared to unblocked condition. In vitro studies showed killing of CSF1R+ cells when engineered exosomes plus splenocytes (from tumor bearing animals) were added to the cells. In vivo studies showed relatively inhibition of tumor growth (compared to control exosome treated animals) when engineered exosomes were administered IV in tumor bearing animals. The data shows that engineered therapeutic CSF1R+ cell targeting exosomes are effective in killing cells and inhibited tumor growth in vivo.

### Example 9: Glioblastoma Targeting Exosome Targets and Kills by ADCC

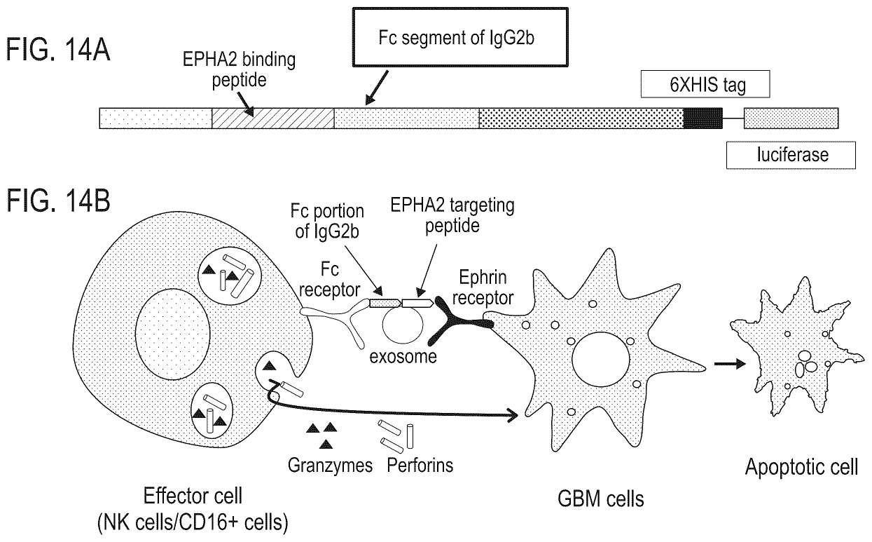

A GBM-targeting exosome was developed using the Fc gamma-receptor (FcR) based targeting exosome platform, wherein the plasmid contained the EPHA2 binding peptide that targets and kills GBM cells by ADCC. FIG. 14A is a schematic representation of the modified Lamp2b protein containing EPHA2-targeting peptide sequence, the Fc segment of IgG2b, following signal peptide, and a 6×HIS tag as shown below.

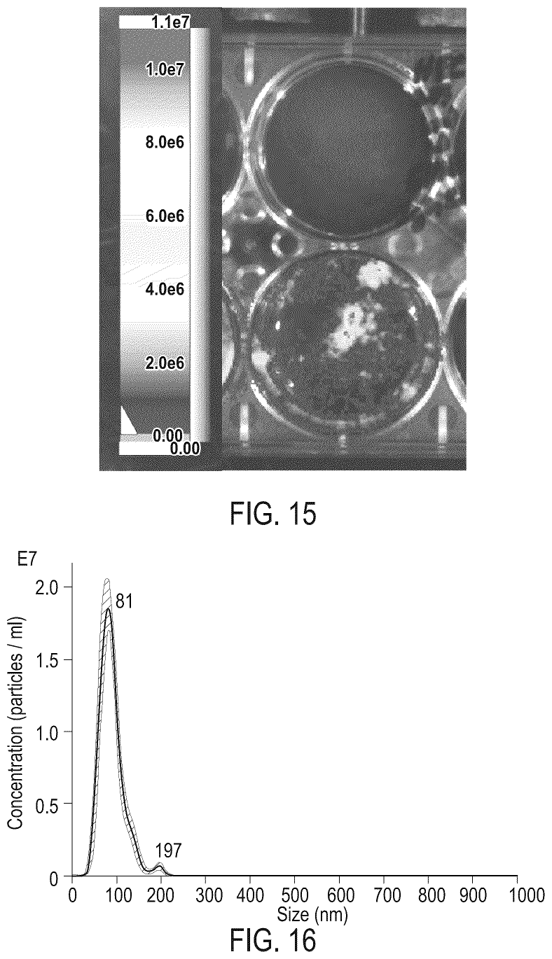

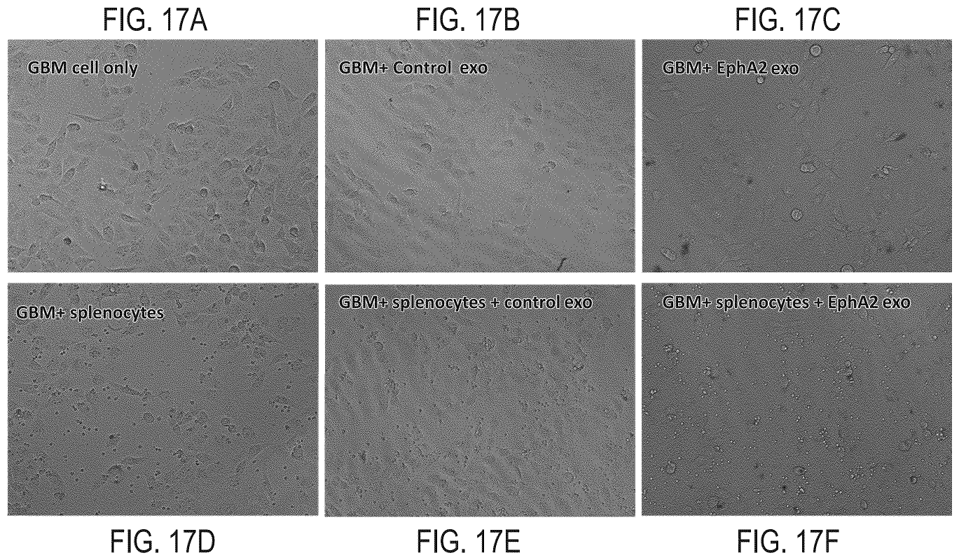

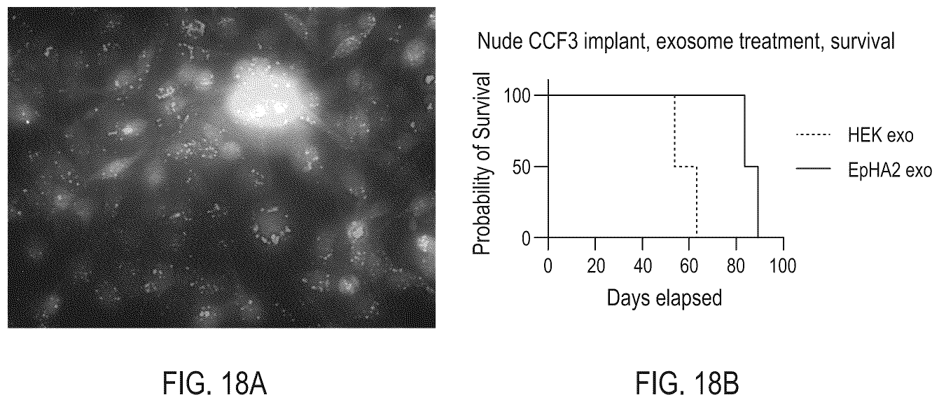

Lamp2b signal peptide-linker-EPHA2 targeting peptide (TACAGCGCCTACCCCGACAGCGTGCCCATGATGAGC (SEQ ID NO: 11))-linker-mIgG2b-linker-Lamp2b remaining sequence-His-tag-Luciferase. The plasmid was stably transfected into HEK293 cells (FIG. 15) and expressed at a high concentration (FIG. 16) showing the size distribution of engineered exosomes targeting EpHA2+ cells using NTA (nanoparticle tracking analysis) instrument. Treatment with EpHA2 targeting exosomes carrying Fc-mIgG2b depleted GBM cells in vitro (FIGS. 17A-17F) and in vivo survival (FIG. 18B). FIG. 18A shows the attachment of the engineered exosomes on the surface of the GBM cells.

### Example 10: Neutrophil Targeting Exosome Targets and Kills by ADCC

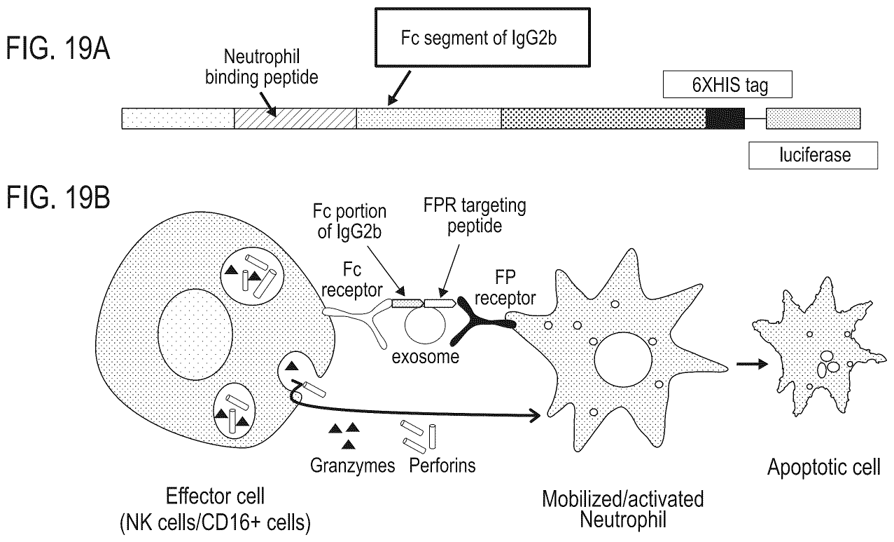

Neutrophil-targeting exosomes were developed using the Fc gamma-receptor (FcR) based targeting exosome platform as shown below and in FIG. 19A.

Lamp2b signal peptide-linker-FTFEPF (SEQ ID NO: 12) antagonistic peptide (TTCACCTTCGAGCCCTTC (SEQ ID NO:13))-linker-mIgG2b-linker-Lamp2b remaining sequence-His-tag-Luciferase.

Lamp2b signal peptide-linker-ETYIKPWWVWL (SEQ ID NO: 14) antagonistic peptide (GAGACCTACATCAAGCCCTGGTGGGTGTGGCTG (SEQ ID NO:15))-linker-mIgG2b-linker-Lamp2b remaining sequence-His-tag-Luciferase

Lamp2b signal peptide-linker-FTFEPF (SEQ ID NO: 12) antagonistic peptide (TTCACCTTCGAGCCCTTC (SEQ ID NO:13) linker ETYIKPWWVWL (SEQ ID NO: 14) antagonistic peptide (GAGACCTACATCAAGCCCTGGTGGGTGTGGCTG (SEQ ID NO:15))-linker-mIgG2b-linker-Lamp2b remaining sequence-His-tag-Luceferase

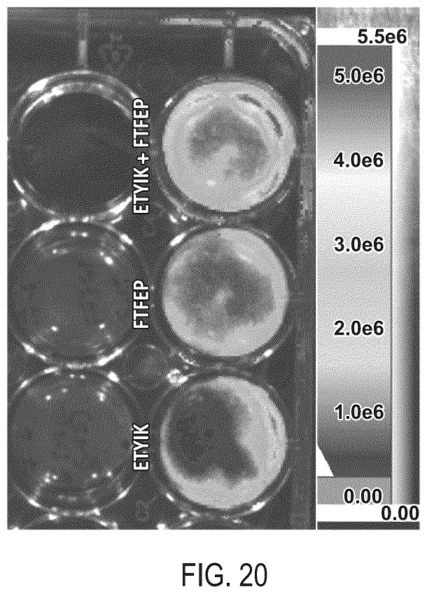

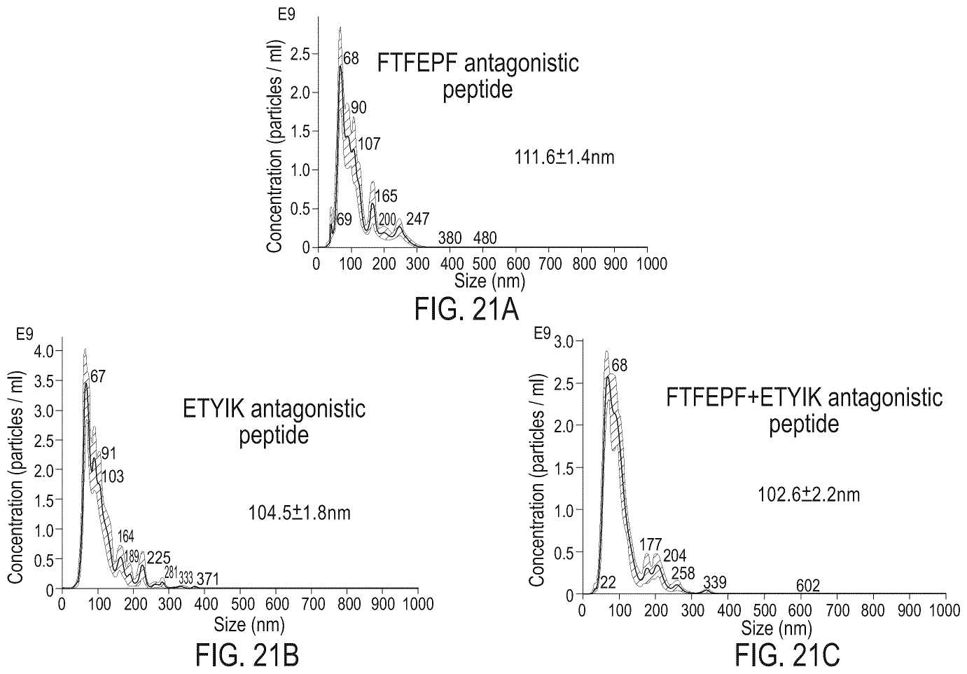

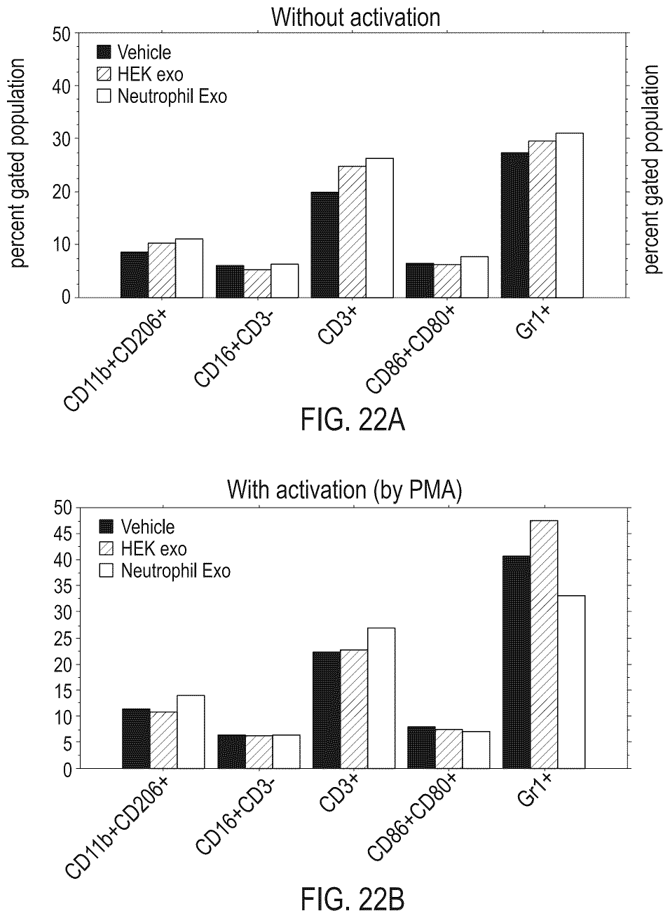

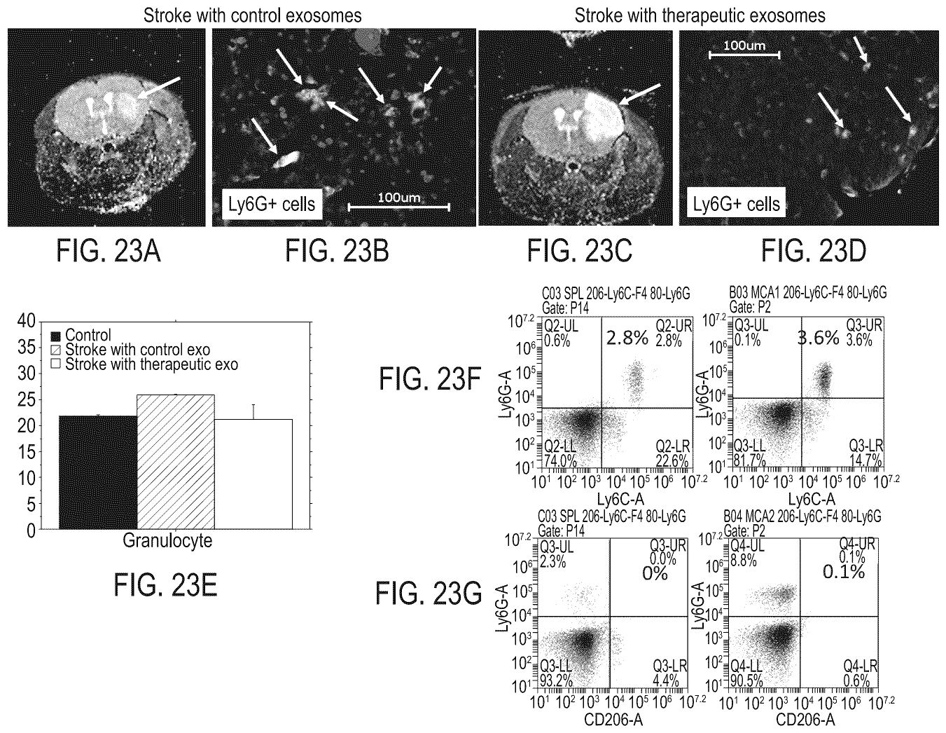

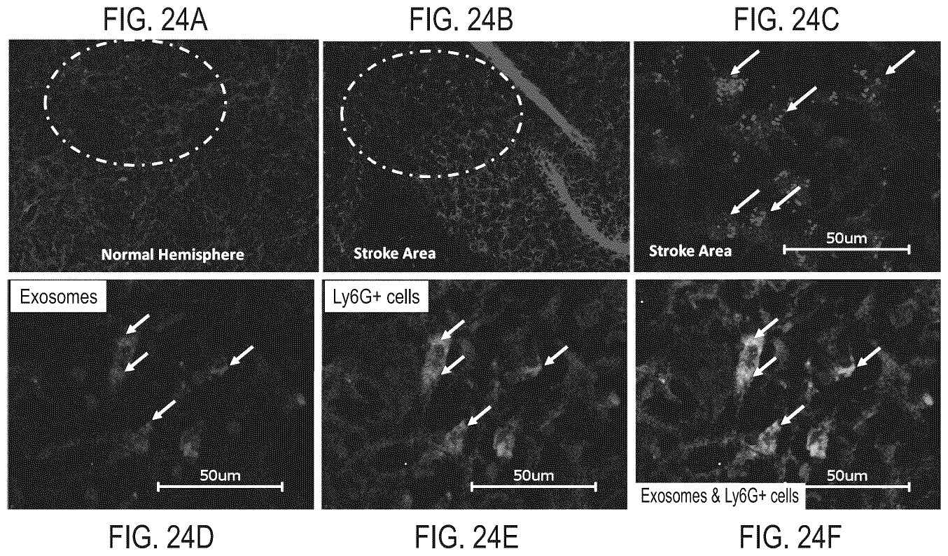

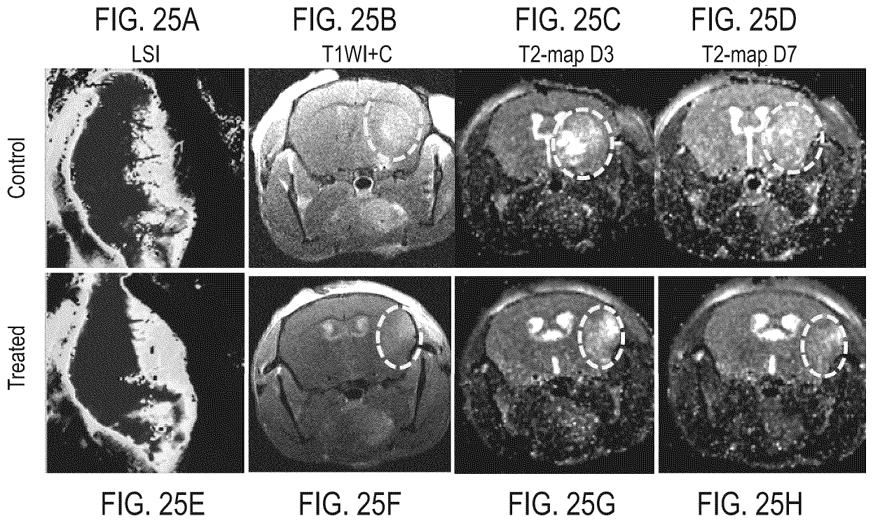

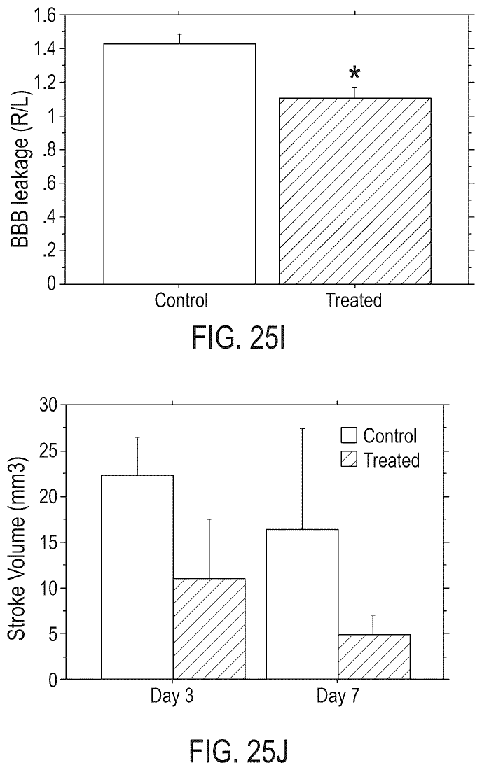

The plasmids were stably transfected into HEK293 and showed successful transfection (FIG. 20). In vitro (FIGS. 22A-22B) and in vivo (FIGS. 23A-23G) studies showed depletion of activated neutrophils. Moreover, the therapeutic neutrophil targeting exosomes were delivered in vivo to the site of stroke through BBB targeting granulocytes (FIGS. 24A-23F). Depleting neutrophils using the engineered exosomes decreased stroke volume (FIGS. 25A-25H and 25J) as well as BBB leakage (FIG. 25I).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An engineered targeting exosome comprising;
a. a Lamp2b signal peptide;
b. a targeting peptide or antibody;
c. a Fc portion of IgG2b
c. a c-terminus portion of Lamp2b protein;
d. a tag protein at the C-terminus; and
e. a reporter gene;
wherein, the targeting peptide or antibody detects target protein within cells expressing the target protein and precisely delivers the exosome to the cells expressing the target protein; and
wherein the Fc portion of IgGb2 induces antibody-dependent cell-mediated cytotoxicity (ADCC) events in cells expressing the target protein.

2. The engineered targeting exosome of claim 1, wherein the targeting peptide or antibody peptide or antibody that is attached to the lumen or the surface of an exosome.

3. The engineered targeting exosome of claim 1, wherein the targeting peptide or antibody is a fusion peptide or antibody comprises a flag tag, a therapeutic peptide, a targeting moiety, or other peptide or antibody attached to the peptide or antibody, or a modification or a fragment of the peptide or antibody.

4. The engineered targeting exosome of claim 1, wherein the targeting peptide or antibody targets proteins involved in tumorigenesis and neurological disorders.

5. The engineered targeting exosome of claim 1, wherein the engineered targeting exosome penetrates through the blood brain barrier to target cells wherein the target protein is expressed.

6. The engineered targeting exosome of claim 1, wherein the targeting peptide or antibody targets proteins selected from a group consisting of CD206+ M2-macrophage, rabies virus glycoprotein (RVG), EPHA2, CSF-1R, and neutrophils (FPRs).

7. The engineered targeting exosome of claim 1, wherein the targeting peptide or antibody targets protein is encoded by nucleic acid sequences with 95%, 99% or more sequence identity to SEQ ID NO:2, 8, 9, 10, 11, 12 or 13.

8. The engineered targeting exosome of claim 1, wherein the Lamp2b signal peptide is encoded by a nucleic acid sequence with 95%, 99% or more sequence identity to SEQ ID NO:4 and the modified Lamp2b protein is encoded by a nucleic acid sequence with 95%, 99% or more sequence identity to SEQ ID NO:5.

9. The engineered targeting exosome of claim 1, wherein the Fc portion of IgG2b is encoded by a nucleic acid sequence with 95%, 99% or more sequence identity to SEQ ID NO:6.

10. The engineered targeting exosome of claim 1, wherein the targeting exosome is loaded with cargo.

11. The engineered targeting exosome of claim 10, wherein the cargo is selected from the group consisting of a detectable label, a chemotherapeutic agent, and a cytotoxic agent.

12. A pharmaceutical composition comprising the engineered targeting exosome of claim 1 and a pharmaceutically acceptable excipient.

13. A method of depleting cells expressing a targeted protein in a subject in need thereof comprising administering to the subject an effective amount of a composition comprising an exosome engineered to express a targeting peptide or antibody and an Fc portion of IgGb2,
wherein the targeting peptide or antibody targets cells expressing the target protein; and
wherein the Fc portion of IgGb2 induces antibody-dependent cell-mediated cytotoxicity (ADCC) in the cells expressing the targeted protein.

14. The method of claim 13, wherein the subject is a mammal.

15. The method of claim 13, wherein the subject has cancer or a neurological disorder.

16. The method of claim 13, wherein the exosomes are loaded with cargo.

17. The method of claim 16, wherein the cargo is selected from the group consisting of a detectable label, a chemotherapeutic agent, and a cytotoxic agent.

18. A method for detecting targeted protein in cells expressing the targeted protein comprising:
contacting a biological sample with a composition comprising an exosome engineered to express a targeting peptide or antibody, wherein the exosome is loaded with a detectable label; and
detecting the detectable label, wherein the detection of the label indicates the presence of the of the targeted protein in the sample;
wherein the exosome comprises a Lamp2b signal peptide; a targeting peptide or antibody; a c-terminus portion of Lamp2b protein; a tag protein at the C-terminus;
and a reporter gene.

19. The method of claim 18, wherein the cells expressing the targeted protein are cancer cells or brain cells.

20. The method of claim 18, wherein the exosome penetrates through the blood brain barrier to treat neurological disorders where the targeted protein is expressed.
